SEQUENCE LISTING (2 sequences)

SEQ ID NO: 1 - DNA, 14988 bp, synthetic construct
  misc_feature 1..14988: note = MEDI RSV DM2-2
  source 1..14988: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaacggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaagacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaggatcaacttctgtcatccagcaaatacgccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagtcaacacatagcattcatcaatccaacagcccaaaacagtaaccttgcatttaaaaatgaacaacccctacctctttacaacacctcattaacatcccaccatgcaaaccactatccatactataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaagccatgaccaacttaaacagaatcaaagtaaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcattgaaactctcatctataaaccatctcacttacactatttaagtagattcctagtttatagttatataaaacacaattgaatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccgcatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaataagcttcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaacactagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 2 - DNA, 14804 bp, synthetic construct
  misc_feature 1..14804: note = rA2 cp248/404/1030/deltaSH
  source 1..14804: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcgattaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatattaaggaaaataagtgtaatggaacagatgctaaggtaaaattgataaaacaagaattagataaatataaaaatgctgtaacagaattgcagttgctcatgcaaagtactccagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatcgcgactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccattaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaacatgtcgcgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcctaaggtgcggattcaataatgttatcttgacacaactattcctttatggagattacatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaacgcatgctctggcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacgtatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccactcgagtgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggctaacaaatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtttaaacgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctgttaactaaatacataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcggaccgtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtcggtaaccgtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttacgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

(19) United States
(12) Patent Application Publication
(10) Pub. No.: US 2026/0091105 A1
(43) Pub. Date: Apr. 2, 2026

(54) METHOD OF VACCINATION WITH AN ATTENUATED RSV VACCINE FORMULATION

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: Peter L. COLLINS, Silver Spring, MD (US); Ursula J. BUCHHOLZ, Silver Spring, MD (US)

(21) Appl. No.: 18/898,953

(22) Filed: Sep. 27, 2024

(51) Int. Cl. A61K 39/155; A61K 9/00; A61K 39/00; A61P 31/14; C12N 7/00

(52) U.S. Cl. CPC A61K 39/155; A61K 9/0043; A61P 31/14; C12N 7/00; A61K 2039/5254; A61K 2039/543; A61K 2039/545; A61K 2039/55; A61K 2039/575; C12N 2760/18534; C12N 2760/18571

(57) ABSTRACT

Respiratory syncytial virus (RSV) infection may lead to severe respiratory illness in young children. Thus, there is a need for a live attenuated vaccine, which would mimic the natural course of infection without causing illness; however, restricting viral replication also reduces the immune response. Reported herein is a method of vaccination using a single dose of a recombinant RSV lacking the M2-2 protein that surprisingly induced a stronger immune response to RSV than previous vaccine candidates despite being more restricted in replication.

### CROSS REFERENCE TO RELATED APPLICATIONS

This application is a continuation of U.S. patent application Ser. No. 15/773,653, filed 4 May 2018, which is a national stage application under 35 U.S.C. 371 and claims the benefit of PCT Application No. PCT/US2016/060672 having an international filing date of 4 Nov. 2016, which designated the United States, which PCT application claimed priority to U.S. Provisional Application Ser. No. 62/251,030, filed Nov. 4, 2015, U.S. Provisional Application Ser. No. 62/259,472, filed Nov. 24, 2015, and U.S. Provisional Application Ser. No. 62/263,405, filed Dec. 4, 2015, all of which are incorporated herein by reference in their entireties for all purposes.

### GOVERNMENT RIGHTS

The Government of the United States has certain rights in this invention.

### SEQUENCE LISTING

This application contains a Sequence Listing submitted as an electronic XML file named “3000094-010004_Sequence Listing.xml”, having a size in bytes of 33,433 bytes, and created on Sep. 25, 2024. The information contained in this electronic file is hereby incorporated by reference in its entirety pursuant to 37 CFR § 1.52 (e) (5).

### TECHNICAL FIELD

The present invention generally relates to methods and compositions useful in vaccinating against respiratory syncytial virus (RSV).

### BACKGROUND

Respiratory syncytial virus (RSV) is the most important viral cause of severe acute lower respiratory illness (LRI) in infants and children worldwide. In the United States, RSV is the leading cause of hospitalization in children less than 1 year of age and is associated with a considerable burden of emergency room and outpatient care, with 10% of children less than 5 years of age receiving medical attention for RSV associated illness each year. Globally, RSV has been estimated to cause >3.5 million hospitalizations and 66,000 to 199,000 deaths annually. The relative importance of RSV as a pulmonary pathogen has also increased because the use of vaccines to prevent bacterial pneumonias has become widespread.

RSV is a member of the Pneumoviridae family and, as such, is an enveloped virus that replicates in the cytoplasm and matures by budding at the host cell plasma membrane. The genome of RSV is a single, negative-sense strand of RNA of 15.2 kilobases that is transcribed by the viral polymerase into 10 mRNAs by a sequential stop-start mechanism that initiates at a single viral promoter at the 3′ end of the genome. Each mRNA encodes a single major protein, with the exception of the M2 mRNA that has two overlapping open reading frames (ORFs) encoding two separate proteins M2-1 and M2-2. The 11 RSV proteins are: the RNA-binding nucleoprotein (N), the phosphoprotein (P), the large polymerase protein (L), the attachment glycoprotein (G), the fusion protein (F), the small hydrophobic (SH) surface glycoprotein, the internal matrix protein (M), the two nonstructural proteins NS1 and NS2, and the M2-1 and M2-2 proteins. The RSV gene order is: 3′-NS1-NS2-N-P-M-SH-G-F-M2-L. Each gene is flanked by short conserved transcription signals called the gene-start (GS) signal, present on the upstream end of each gene and involved in initiating transcription of the respective gene, and the gene-end (GE) signal, present at the downstream end of each gene and involved in directing synthesis of a polyA tail followed by release of the mRNA.

The RSV F and G proteins are the only RSV proteins known to induce RSV neutralizing antibodies, and are the major protective antigens. The F protein generally is considered to be a more effective neutralization and protective antigen than the G protein. F also is relatively well-conserved among RSV strains, whereas the G protein can be substantially divergent. The divergence in G is a major factor in segregating RSV strains into two antigenic subgroups, A and B (˜53% and ˜90% amino acid sequence identity between the two subgroups for G and F, respectively).

Because of the substantial global impact of RSV, increased efforts are under way to develop RSV vaccines for use in infancy and early childhood. A live attenuated RSV vaccine would be an attractive strategy for immunization of children and infants. Live attenuated vaccines mimic a mild natural infection and induce durable cellular and humoral immune responses. Furthermore, administration of candidate live attenuated RSV vaccines is not associated with the vaccine-associated enhanced RSV disease that was observed in children who received formalin-inactivated RSV and that also appeared to be associated with administration of RSV subunit vaccines in experimental animals.

Efforts to develop live attenuated RSV vaccines have been underway since the 1970s. The process of attenuation has been challenging because conventional methods, such as passage of virus at suboptimal temperatures or in the presence of mutagens, are targeted imprecisely and are poorly controlled. In addition, clinical attenuation typically is based on restriction of replication, which decreases antigenic load and diminishes the immune response. In the past, achieving a balance between attenuation and immunogenicity has proved difficult: some live attenuated RSV vaccine candidates have been insufficiently attenuated, whereas others were highly attenuated but insufficiently immunogenic. In addition, some candidate vaccines have exhibited genetic instability, with a partial loss of attenuating mutations. Thus, a need exists in the art to provide improved methods of vaccination against RSV.

### SUMMARY

In one embodiment of the invention, it includes a method of vaccinating a human subject against respiratory syncytial virus (RSV), comprising administering to the subject a composition comprising an RSV particle that comprises an RSV genome or antigenome, wherein the subject is less than about 24 months of age, wherein the composition is administered in a single dose, and wherein the RSV genome or antigenome comprises a functional deletion in the M2-2 ORF corresponding to a deletion of nucleotides 8201-8434 in Genbank Accession No. M74568; and optionally comprises a characteristic selected from the group consisting of: (i) presence of one or more of, corresponding to the nucleotides in Genbank Accession No. M74568, C4488 in the SH gene, C4491 in the SH gene, A4494 in the SH gene, A4496 in the SH gene, G4497 in the SH gene, and a nucleotide sequence corresponding to nucleotides 4498-4609 in the non-translated region of the SH gene; (ii) presence of G at a nucleotide position corresponding to position 1209 in Genbank Accession No. M74568 encoding an Alanine at position 24 in the N protein; (iii) presence of G at a nucleotide position corresponding to position 779 in Genbank Accession No. M74568 encoding an Arginine at position 51 in the NS2 protein; (iv) presence of one or more nucleotides corresponding to the nucleotides in Genbank Accession No. M74568 selected from the group consisting of: A1138, G1139, A5611, A5615, G5639, T7481 and A7559; (v) presence of one or more nucleotides corresponding to the nucleotides in Genbank Accession No. M74568 selected from the group consisting of: C404 in the NS1 gene, G1181 in the N gene, A1937 in the N gene, C6215 in the F gene, C6221 in the F gene, T6386 in the F gene, C7214 in the F gene, G7701 in the M2 gene; T8160 in the M2-2 gene, T8166 in the M2-2 gene, T8178 in the M2-2 gene, A8197 in the M2-2 gene, G8198 in the M2-2 gene, T 10514 in the L gene, A 13633 in the L gene, and T 13900 in the L gene; (vi) presence of T at a nucleotide position corresponding to position 1099 in Genbank Accession No. M74568; and (vii) a combination of the above.

In some embodiments, the single dose of the composition comprises about 10^{3.0} to about 10^{7.0} plaque-forming units (PFU) of RSV particles. In some embodiments, the single dose of the composition comprises about 10^{4.0} to about 10^{6.0} plaque-forming units (PFU) of RSV particles. In some embodiments, the single dose of the composition comprises about 10^{4.0} plaque-forming units (PFU) of RSV particles. In some embodiments, single dose of the composition comprises about 10^{5.0} plaque-forming units (PFU) of RSV particles. In some embodiments, the single dose of the composition comprises about 10^{6.0} plaque-forming units (PFU) of RSV particles.

In some embodiments, the single dose is administered in about 0.2 ml to about 1 ml total volume per subject. In some embodiments, the single dose is administered in about 0.5 ml total volume per subject.

In some embodiments, the RSV genome or antigenome comprises at least one characteristic listed in (i)-(vi). In some embodiments, the RSV genome or antigenome comprises all characteristics listed in (i)-(vi).

In some embodiments, the RSV genome or antigenome comprises a nucleotide sequence that is at least 90% identical to SEQ ID NO:1. In some embodiments, the RSV genome or antigenome comprises the nucleotide sequence of SEQ ID NO:1.

In some embodiments, the RSV particle is live attenuated. In some embodiments, the RSV genome or antigenome comprises one or more dimorphisms selected from the group consisting of: (i) A/G dimorphism at nucleotide position 285 in the NS1 gene resulting in a mixture of amino acid assignments S/G at amino acid position 63 (ii) C/T dimorphism at nucleotide position 900 in the NS2 gene; and (iii) T/G dimorphism at nucleotide position 4311 in the SH gene resulting in a mixture of amino acid assignments N/K at amino acid position 3.

In some embodiments, the subject is less than about 18 months of age. In some embodiments, the subject is less than about 12 months of age. In some embodiments, the subject is less than about 6 months of age. In some embodiments, the subject is less than about 4 months of age. In some embodiments, the subject is less than about 2 months of age.

In some embodiments, the route of administration is intranasal, intramuscular or subcutaneous. In some embodiments, the route of administration is intranasal. In some embodiments, the composition is administered via nasal drops. In some embodiments, the composition is administered via nasal spray. In some embodiments, the composition is administered via nasal powder.

In some embodiments, the RSV particle exhibits restricted replication in the subject, In some embodiments, the RSV particle exhibits enhanced immunogenicity in the subject, In some embodiments, the subject exhibits anamnestic response to wild type RSV infection.

In some embodiments, the invention includes a method for vaccinating a human subject against respiratory syncytial virus (RSV) infection, comprising administering to the subject a composition comprising an RSV particle that comprises an RSV genome or antigenome, wherein the subject less than about 24 months of age, wherein the composition is administered intranasally in a single dose, and wherein the RSV genome or antigenome comprises: (i) a functional deletion in the M2-2 ORF corresponding to a deletion of nucleotides 8201-8434 in Genbank Accession No. M74568; (ii) presence of, corresponding to the nucleotides in Genbank Accession No. M74568, C4488 in the SH gene, C4491 in the SH gene, A4494 in the SH gene, A4496 in the SH gene, G4497 in the SH gene, and a nucleotide sequence corresponding to nucleotides 4498-4609 in the non-translated region of the SH gene; (iii) presence of G at a nucleotide position corresponding to position 1209 in Genbank Accession No. M74568 encoding an Alanine at position 24 in the N protein; (iv) presence of G at a nucleotide position corresponding to position 779 in Genbank Accession No. M74568 encoding an Arginine at position 51 in the NS2 protein; (v) presence of nucleotides corresponding to the nucleotides in Genbank Accession No. M74568 selected from the group consisting of: A1138, G1139, A5611, A5615, G5639, T7481 and A7559; (vi) presence of nucleotides corresponding to the nucleotides in Genbank Accession No. M74568 selected from the group consisting of: C404 in the NS1 gene, G1181 in the N gene, A1939 A1937 in the N gene, C6215 in the F gene, C6221 in the F gene, T6386 in the F gene, C7214 in the F gene, G7701 in the M2 gene; T8160 in the M2-2 gene, T8166 in the M2-2 gene, T8178 in the M2-2 gene, A8197 in the M2-2 gene, G8198 in the M2-2 gene; T 10514 in the L gene, A 13633 in the L gene, and T 13900 in the L gene and (vii) presence of T at a nucleotide position corresponding to position 1099 in Genbank Accession No. M74568. In some embodiments, the RSV genome or antigenome comprises the nucleotide sequence of SEQ ID NO:1.

In some embodiments, the single dose of the composition comprises about 10⁵ plaque-forming units (PFU) or less of the RSV particle. In some embodiments, the single dose of the composition comprises about 10⁶ plaque-forming units (PFU) or less of the RSV particle. In some embodiments, the single dose of the composition comprises about 10⁴ plaque-forming units (PFU) or less of the RSV particle.

### BRIEF DESCRIPTION OF THE DRAWINGS

FIG. 1 is a schematic depiction of the screening, enrollment, and follow-up of RSV-seropositive children (A) and RSV-seronegative children (B) in the phase 1 clinical trial of the RSV MEDI ΔM2-2 vaccine. As described in the text, the study was initiated with administration of the RSV MEDI Δ M2-2 vaccine to 15 adults in an open label evaluation. For this portion of the study, 31 adults were screened, 19 were eligible, 15 were enrolled, and none were lost to follow-up.

FIG. 2 shows the proportions of RSV-seronegative vaccinees and placebo recipients with indicated illnesses. The placebo recipient (twin B) infected with vaccine virus is not included. Fever occurred in 4 of 20 vaccinees (1 of grade 0 severity, 1 of grade 2 severity, and 2 of grade 3 severity) and in 2 of 9 placebo recipients (both of grade 2 severity): grade 0, <38° C.; grade 1, ≥38° C. but ≤38.6° C.; grade 2, ≥38.7° C. but ≤39.1° C.; grade 3, ≥39.2° C. but ≤40.5° C. The two episodes of grade 3 fever in vaccinees occurred on days 24 to 26 after vaccination, when shedding of vaccine virus was not detected. URI (rhinorrhea) occurred in 17 of 20 vaccinees and 4 of 9 placebo recipients; cough occurred in 7 of 20 vaccinees and 3 of 9 placebo recipients; all of these illnesses were of grade 1 severity (that is, not requiring medical attention). An episode of otitis media (OM, grade 2 severity) occurred in a single vaccinee.

FIG. 3 shows vaccine virus shedding and serum RSV neutralizing antibody responses in seronegative recipients of RSV MEDI ΔM2-2 or rA2 cp248/404/1030/ΔSH. NW and serum specimens from the present study were evaluated for vaccine virus titer and serum neutralizing antibodies, respectively. Antibody testing was performed in parallel with specimens from the previous clinical evaluation of rA2 cp248/404/1030/ΔSH. (A) Peak viral titer (expressed as log₁₀ PFU/ml). (B) RSV Plaque Reduction Neutralization Titers (PRNTs), expressed as 1/log₂. * P=0.005; +P=0.002 (Student's t test).

FIG. 4 shows the reverse cumulative distribution of RSV PRNT in seronegative vaccinees. The solid line represents vaccinees, and the dashed line represents placebo recipients. Antibody titers are expressed as 1/log₂, but for ease of interpretation, the titer corresponding to the arithmetic value of 1:64, achieved by 85% of vaccinees, is also shown.

FIG. 5. Rises in RSV PRNT during the surveillance period. RSV PRNT in sera from six vaccinees (filled circles) and three placebo recipients (open triangles) in whom ≥4-fold rises in titer between the pre- and post-surveillance specimens were detected (see text for additional details). Solid black lines, vaccinees with rises in RSV PRNT in whom no RSV-associated Medically Attended Acute Respiratory Illness (RSV MAARI) was detected (n=5; corresponding to subjects 33, 35, 44, 46, and 56 in the supplementary tables); dashed black line, vaccinee with rise in RSV PRNT in whom RSV MAARI was detected (n=1; subject 38 in the supplementary tables); solid grey line, placebo recipient with rise in RSV PRNT in whom no RSV MAARI was detected (n=1; subject 36 in the supplementary tables); dashed grey lines, placebo recipients with rise in RSV PRNT in whom RSV MAARI was detected (n=2; subjects 43 and 57 in the supplementary tables). Antibody titers are expressed as 1/log₂, but for ease of interpretation, titers corresponding to the arithmetic values of 1:16, 1:64, 1:256, 1:1024, and 1:4096 are also shown.

FIG. 6 shows the nucleotide sequence of the RSV MEDI ΔM2-2 recombinant virus (SEQ ID NO:1).

FIG. 7 shows the nucleotide sequence of the RSV rA2 cp248/404/1030ΔSH recombinant virus (SEQ ID NO:2).

### DETAILED DESCRIPTION

Reported herein is a method of vaccinating a human subject against Respiratory Syncytial Virus (RSV) by administering a composition comprising an immunogenic amount of a recombinant RSV particle described below to the subject. An embodiment of the composition comprising the recombinant RSV particle was evaluated as a live intranasal vaccine in adults, RSV-seropositive children and RSV-seronegative children. When results in RSV-seronegative children were compared to those achieved with the previous leading live attenuated RSV candidate vaccine, vaccine virus shedding was significantly more restricted, yet the post-vaccination RSV-neutralizing serum antibody achieved was significantly greater. Surveillance during the subsequent RSV season showed that several RSV-seronegative recipients had substantial rises of RSV-neutralizing serum antibodies indicative of exposure to RSV, and yet without reported RSV-associated illness, suggesting that the vaccine was protective yet primed for anamnestic responses to RSV. Thus, the composition comprising the recombinant RSV particle was intrinsically superior at eliciting protective antibody in the subjects. Surprisingly, a single dose of the composition was sufficient to provide the greater antibody response and protective effect in seronegative and/or RSV-naïve infants and children of less than about 24 months of age. This was an unexpected result, as it is currently anticipated that vaccination against RSV using a live, attenuated RSV vaccine will require administration of multiple doses, at least two or three at a minimum, in a single vaccination season to provide protective result.

Thus, in one embodiment, the invention includes a method of vaccinating a human subject to protect against RSV infection, or inducing a protective immune response in a human subject against RSV or stimulating the immune system of a human subject to elicit an immune response against RSV. The method comprises administering to the subject an immunogenic composition comprising an immunologically sufficient amount of an RSV particle.

In some embodiments of the invention, the RSV particle comprises an RSV genome or antigenome in which the RSV genome or antigenome contains a functional deletion in the M2-2 Open Reading Frame (ORF) which results in the ablation of the M2-2 protein, optionally further coupled with one or more characteristics as described in the table below. Without wishing to be bound by theory, the superior performance of the RSV particle is believed to be, at least in part, due to one or more of these characteristics.

| | RSV Nucleotide | | Amino Acid | |
|---|---|---|---|---|
| Gene | Genomic nt | | Amino Acid | |
| Region | position¹ | cDNA | Position | cDNA |
| NS1 | 404 | C | 102 | N |
| NS2 | 779 | G | 51 | R |
| NS2/N ig² | 1099 | T | ncr³ | — |
| N | 1138 | A | ncr³ | — |
| N | 1139 | G | ncr³ | — |
| N | 1181 | G | 14 | K |
| N | 1209 | G | 24 | A |
| N | 1937 | A | 266 | S |
| SH | 4488 | C | 62 | V |
| SH | 4491 | C | 63 | N |
| SH | 4494 | A | 64 | T |
| SH | 4496 | A | 65 | stop |
| SH | 4497 | G | 65 | stop |
| SH | 4498-4609 | No deletion | ncr³ | — |
| G/F ig² | 5611 | A | ncr³ | — |
| G/F ig² | 5615 | A | ncr³ | — |
| G/F ig² | 5639 | G | ncr³ | — |
| F | 6215 | C | 185 | V |
| F | 6221 | C | 187 | V |
| F | 6386 | T | 242 | G |
| F | 7214 | C | 518 | A |
| F | 7481 | T | ncr³ | — |
| F/M2 ig² | 7559 | A | ncr³ | — |
| M2 | 7701 | G | 32 | P |
| M2-2 | 8160 | T | 1 | M |
| M2-2 | 8166 | T | 3 | M |
| M2-2 | 8178 | T | 7 | M |
| M2-2 | 8197 | A | 13 | stop |
| M2-2 | 8198 | G | ncr³ | — |
| M2-2 | 8201-8434 | 234 nt | | M2-2 del |
| | | deletion | | |
| L | 10514 | T | 673 | L |
| L | 13633 | A | 1712 | T |
| L | 13900 | T | 1801 | S |

¹Genomic position numbered relative to WT RSV strain A2 (Genbank accession number M74568). All sequences are positive-sense.
²ig, intergenic region
³ncr, non-coding region

The numbering used in this disclosure is based on the sequence of the wild-type RSV A2 strain (Genbank accession number M74568) and all sequences described are in positive-sense. Given that a variety of RSV strains exist (e.g., RSV A2, RSV B1, RSV Long), those skilled in the art will appreciate that certain strains of RSV may have nucleotide or amino acid insertions or deletions that alter the position of a given residue. For example, if a protein of another RSV strain had, in comparison with strain A2, two additional amino acids in the upstream end of the protein, this would cause the amino acid numbering of downstream residues relative to strain A2 to increase by an increment of two. However, because these strains share a large degree of sequence identity, those skilled in the art would be able to determine the location of corresponding sequences by simply aligning the nucleotide or amino acid sequence of the A2 reference strain with that of the strain in question. Therefore, it should be understood that the amino acid and nucleotide positions described herein, though specifically enumerated in the context of this disclosure, can correspond to other positions when a sequence shift has occurred or due to sequence variation between virus strains. In the comparison of a protein, or protein segment, or gene, or genome, or genome segment between two or more related viruses, a “corresponding” amino acid or nucleotide residue is one that is thought to be exactly or approximately equivalent in function in the different species.

The wild type RSV virus genome or antigenome encodes the following 11 proteins: the RNA-binding nucleoprotein (N), the phosphoprotein (P), the large polymerase protein (L), the attachment glycoprotein (G), the fusion protein (F), the small hydrophobic (SH) surface glycoprotein, the internal matrix protein (M), the two nonstructural proteins NS1 and NS2, and the M2-1 and M2-2 proteins. The RSV gene order is: 3′-NS1-NS2-N-P-M-SH-G-F-M2-L. The complete amino acid sequences of these proteins, and nucleotides encoding them are known in the art.

In some embodiments of the present invention, the RSV genome or antigenome comprises a functional deletion in the M2-2 ORF which comprises a deletion of 234 nt corresponding to positions 8201-8434 encoding the C-terminal 78 amino acids of the M2-2 protein. The N-terminal 12 amino acid residues encoded by the M2-2 open reading frame are maintained at the region of overlap with the M2-1 open reading frame. See Example 1. The deletion in the M2-2 ORF results in the ablation of the M2-2 protein which down-regulates viral RNA replication and up regulates gene transcription and antigen synthesis. This is believed to be the first time that a viral vaccine candidate designed for increased gene transcription has been evaluated in humans.

In some embodiments, the RSV genome or antigenome further comprises one or more of the following characteristics. In some embodiments, the RSV genome or antigenome comprises presence of G at a nucleotide position corresponding to position 779 encoding an Arginine at position 51 in the NS2 protein. In some embodiments, the RSV genome or antigenome comprises the presence of G at a nucleotide position corresponding to position 1209 encoding an Alanine at position 24 in the N protein.

In some embodiments, the RSV genome or antigenome comprises the presence of C at a nucleotide position corresponding to position 404 in the NS1 gene. In some embodiments, the RSV genome or antigenome comprises the presence of T at a nucleotide position corresponding to position 1099 in the NS2/N intergenic region.

In some embodiments, the RSV genome or antigenome comprises the presence of A at a nucleotide position corresponding to position 1138 in the non-coding region of the N gene. In some embodiments, the RSV genome or antigenome comprises the presence of G at a nucleotide position corresponding to position 1139 in the non-coding region of the N gene. In some embodiments, the RSV genome or antigenome comprises the presence of G at a nucleotide position corresponding to position 1181 in the N gene. In some embodiments, the RSV genome or antigenome comprises the presence of A at a nucleotide position corresponding to position 1937 in the N gene.

Bukreyev et al. previously described the 6120 mutation in the SH gene which involves introduction of five translationally-silent point mutations at nucleotide position 4488, 4491, 4494, 4496 and 4497, and deletion of nucleotides 4498-4609 in the downstream non-translated region of the SH gene (Bukreyev et al., 2001. J Virol. 75:12128-12140). In some embodiments, the RSV genome or antigenome does not contain the 6120 mutation. In some embodiments, the RSV genome or antigenome comprises the presence of C at a nucleotide position corresponding to position 4488 in the SH gene. In some embodiments, the RSV genome or antigenome comprises the presence of C at a nucleotide position corresponding to position 4491 in the SH gene. In some embodiments, the RSV genome or antigenome comprises the presence of A at a nucleotide position corresponding to position 4494 in the SH gene. In some embodiments, the RSV genome or antigenome comprises the presence of A at a nucleotide position corresponding to position 4496 in the SH gene. In some embodiments, the RSV genome or antigenome comprises the presence of G at a nucleotide position corresponding to position 4497 in the SH gene. In some embodiments, the RSV genome or antigenome comprises the presence in the non-coding region of the SH gene of a nucleotide sequence corresponding to nucleotides 4498-4609.

In some embodiments, the RSV genome or antigenome comprises the presence of A at a nucleotide position corresponding to position 5611 in the G/F intergenic region. In some embodiments, the RSV genome or antigenome comprises the presence of A at a nucleotide position corresponding to position 5615 in the G/F intergenic region. In some embodiments, the RSV genome or antigenome comprises the presence of G at a nucleotide position corresponding to position 5639 in the G/F intergenic region.

In some embodiments, the RSV genome or antigenome comprises the presence of C at a nucleotide position corresponding to position 6215 in the F gene. In some embodiments, the RSV genome or antigenome comprises the presence of C at a nucleotide position corresponding to position 6221 in the F gene. In some embodiments, the RSV genome or antigenome comprises the presence of T at a nucleotide position corresponding to position 6386 in the F gene. In some embodiments, the RSV genome or antigenome comprises the presence of C at a nucleotide position corresponding to position 7214 in the F gene. In some embodiments, the RSV genome or antigenome comprises the presence of T at a nucleotide position corresponding to position 7481 in the non-coding region of the F gene.

In some embodiments, the RSV genome or antigenome comprises the presence of A at a nucleotide position corresponding to position 7559 in the F/M2 intergenic region.

In some embodiments, the RSV genome or antigenome comprises the presence of G at a nucleotide position corresponding to position 7701 in the M2 gene. In some embodiments, the RSV genome or antigenome comprises the presence of T at a nucleotide position corresponding to position 8160 in the M2 gene. In some embodiments, the RSV genome or antigenome comprises the presence of T at a nucleotide position corresponding to position 8166 in the M2 gene. In some embodiments, the RSV genome or antigenome comprises the presence of T at a nucleotide position corresponding to position 8178 in the M2 gene. In some embodiments, the RSV genome or antigenome comprises the presence of A at a nucleotide position corresponding to position 8197 in the M2 gene. In some embodiments, the RSV genome or antigenome comprises the presence of G at a nucleotide position corresponding to position 8198 in the M2 gene.

In some embodiments, the RSV genome or antigenome comprises the presence of T at a nucleotide position corresponding to position 10514 in the L gene. In some embodiments, the RSV genome or antigenome comprises the presence of A at a nucleotide position corresponding to position 13633 in the L gene. In some embodiments, the RSV genome or antigenome comprises the presence of T at a nucleotide position corresponding to position 13900 in the L gene.

In some embodiments, the RSV genome or antigenome comprises the M2-2 deletion and further comprises at least one of the other characteristics described above. In some embodiments, the RSV genome or antigenome comprises the M2-2 deletion and at least two, or three, or four, or five, or six, or seven, or eight, or nine, or ten, or eleven, or twelve, or thirteen, or fourteen, or fifteen, or sixteen, or seventeen, or eighteen, or nineteen, or twenty, or twenty one, or twenty two, or twenty three, or twenty four, or twenty five, twenty six, or twenty seven, or twenty eight, or twenty nine, or thirty, or thirty one of the additional characteristics described above. In some embodiments, the RSV genome or antigenome comprises the M2-2 deletion and all of the additional characteristics described above.

In some embodiments, the RSV genome or antigenome comprises a functional deletion in the M2-2 ORF corresponding to a deletion of nucleotides 8201-8434 in Genbank Accession No. M74568 and optionally comprises a characteristic selected from the following: i) presence of one or more of, corresponding to the nucleotides in Genbank Accession No. M74568, C4488 in the SH gene, C4491 in the SH gene, A4494 in the SH gene, A4496 in the SH gene, G4497 in the SH gene, and a nucleotide sequence corresponding to nucleotides 4498-4609 in the non-translated region of the SH gene, ii) presence of G at a nucleotide position corresponding to position 1209 in Genbank Accession No. M74568 encoding an Alanine at position 24 in the N protein, iii) presence of G at a nucleotide position corresponding to position 779 in Genbank Accession No. M74568 encoding an Arginine at position 51 in the NS2 protein, iv) presence of one or more nucleotides corresponding to the following nucleotides in Genbank Accession No. M74568 selected from the group consisting of: A1138, G1139, A5611, A5615, G5639, T7481 and A7559, v) presence of one or more nucleotides corresponding to the following nucleotides in Genbank Accession No. M74568 selected from the group consisting of: C404 in the NS1 gene, G1181 in the N gene, A1937 in the N gene, C6215 in the F gene, C6221 in the F gene, T6386 in the F gene, C7214 in the F gene, G7701 in the M2 gene; T8160 in the M2-2 gene, T8166 in the M2-2 gene, T8178 in the M2-2 gene, A8197 in the M2-2 gene, and G8198 in the M2-2 gene, vi) presence of T at a nucleotide position corresponding to position 1099 in Genbank Accession No. M74568, and vii) a combination of the above.

In some embodiments, the RSV A2 genome or antigenome comprises the M2-2 deletion and all of the characteristics described in (i)-(vi). In some embodiments, the RSV particle comprises an RSV genome or antigenome comprising the sequence shown in SEQ ID NO: 1. This RSV construct is also referred herein as RSV MEDI ΔM2-2.

Reference to a polypeptide or polynucleotide sequence includes a polypeptide or polynucleotide sequence that is at least about 50%, or at least about 55%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identical (or any percent identity between 45% and 100%, in whole integer increments), to the polypeptide or polynucleotide sequence.

Compared to wild-type RSV, the RSV MEDI ΔM2-2 mutant exhibited a shift in the viral RNA synthesis program such that genome replication was decreased and gene transcription was increased. This resulted in a substantial increase in synthesis of the viral proteins, including the major neutralization and protective antigens, suggesting the possibility of an inherent increase in immunogenicity per infectious unit. In addition, because it is based on a large deletion that ablates expression of a viral protein, the attenuation phenotype of RSV MEDI ΔM2-2 is very stable, obviating a complication of vaccines based on point mutations. In nonhuman primates, RSV MEDI ΔM2-2 was highly restricted in replication but induced substantial neutralizing serum antibody responses and protected against challenge, although it could not be determined whether RSV MEDI ΔM2-2 was inherently more immunogenic than wild-type RSV. On the basis of this preclinical profile of substantial attenuation and immunogenicity, a stepwise phase 1 evaluation of the RSV MEDI ΔM2-2 vaccine candidate was conducted in adults and in RSV-seropositive and RSV-seronegative children, as described in detail in Example 2.

A lot of RSV MEDI ΔM2-2 clinical trial material (CTM) suitable for human evaluation as a live attenuated intranasal vaccine was manufactured. The nucleotide sequence of the RSV MEDI ΔM2-2 CTM was determined and was found to be identical to that of its cDNA clone of origin (SEQ ID NO:1) except for dimorphisms (mixtures of two different nucleotide assignments) at three sequence positions: (i) nucleotide 285, in the NS1 gene, was a mixture of A/G in the CTM compared to A in the cDNA, resulting in a mixture of amino acid assignments S/G at position 63 in the CTM compared to S in the cDNA; (ii) nucleotide 900, in the NS2 gene, was a mixture of C/T, compared to C in the cDNA, with no effect on amino acid coding; and (iii) nucleotide 4311, in the SH gene, was a mixture of T/G in the CTM compared to T in the cDNA, resulting in a mixture of amino acid assignments N/K at amino acid position 3 in the CTM, compared to N in the cDNA.

As described on Example 2, a single dose of a composition comprising the RSV MEDI ΔM2-2 particles was administered to RSV-seronegative infants and children of ages 6-24 months, as well as seropositive children and adults. The term “seronegative” as used herein means an individual (typically but not exclusively ≥6 months of age) who has a serum RSV-neutralizing antibody titer of <1:40 (determined by a complement-enhanced 60% plaque reduction neutralization assay), which typically indicates that the individual had not been previously infected with RSV. The term “RSV-naïve” as used herein means an individual (typically but not exclusively a young infant of <6 months old) who has not been previously exposed to RSV, but potentially might have maternally derived serum RSV-neutralizing antibodies that might have a titer ≥1:40.

In previous vaccine candidates, restriction of viral replication was associated with clinical attenuation, but vaccine candidates that were highly restricted in replication did not induce substantial plaque reduction neutralization titer (PRNT) in RSV-naïve children. Thus, it was difficult to achieve an appropriate balance between attenuation and antibody response in the pediatric target population.

When results in RSV-seronegative children were compared to those achieved with the previous leading live attenuated RSV candidate vaccine, the MEDI ΔM2-2 vaccine virus was significantly more restricted in replication and virus shedding was significantly less, yet the post-vaccination RSV-neutralizing serum antibody achieved (geometric mean titer (GMT)=1:97) was significantly greater. Without wishing to be bound by theory, it is believed that the ΔM2-2 attenuating mutation has produced a vaccine virus that induces a greater antibody response per infectious unit than previous attenuated strains via up-regulation of viral gene transcription and antigen synthesis. It is further believed that that the M2-2 deletion increases RSV-specific T cell responses as a result of increased expression of all viral genes and antigens. MEDI ΔM2-2 also is the first live attenuated RSV vaccine candidate to be evaluated intranasally in young children for which the attenuation does not involve cold-passage (cp) or temperature-sensitivity (ts) mutations, and indeed MEDI ΔM2-2 is not cold-adapted or temperature sensitive compared to wild type RSV. Temperature sensitivity mutations are thought to preferentially restrict viral replication in the lower (warmer) respiratory tract, and cp mutations similarly may restrict replication at increased temperature or favor it at reduced temperature (such as in the upper respiratory tract). These restrictions would not apply to MEDI ΔM2-2 given its lack of ts and cp properties, and it may be that this highly attenuated virus was nonetheless able to replicate in the respiratory tract beyond the upper respiratory tract, which would be thought to be more immunogenic. Therefore, this unusual property also may contribute to the unusual immunogenicity of MEDI ΔM2-2.

Lower respiratory illness (LRI) after administration of this vaccine was not observed, and the rates of fever, cough, and OM were comparable in seronegative vaccinees and placebo recipients. Rhinorrhea occurred in 73% of seronegative children, and more often in seronegative vaccinees (85%) than placebo recipients (44%), although this difference was not statistically significant. Furthermore, the observation (see below) that shedding of infectious vaccine virus was detected in only 60% of vaccinees, and that the mean peak group titer of shed virus irrespective of day was 1.5 log₁₀ PFU/ml of nasal wash (compared to 6.0-7.0 log₁₀ PFU/ml that is often observed with wild type RSV) suggests that this virus is very highly attenuated and unlikely to exhibit reactogenicity. Surveillance during the subsequent RSV season showed that several formerly seronegative RSV MEDI ΔM2-2 recipients had substantial antibody rises without reported illness. Post-vaccination surveillance also provided evidence that some vaccinees had been exposed to wild-type RSV, but had been protected from significant disease due to administration of the RSV MEDI ΔM2-2.

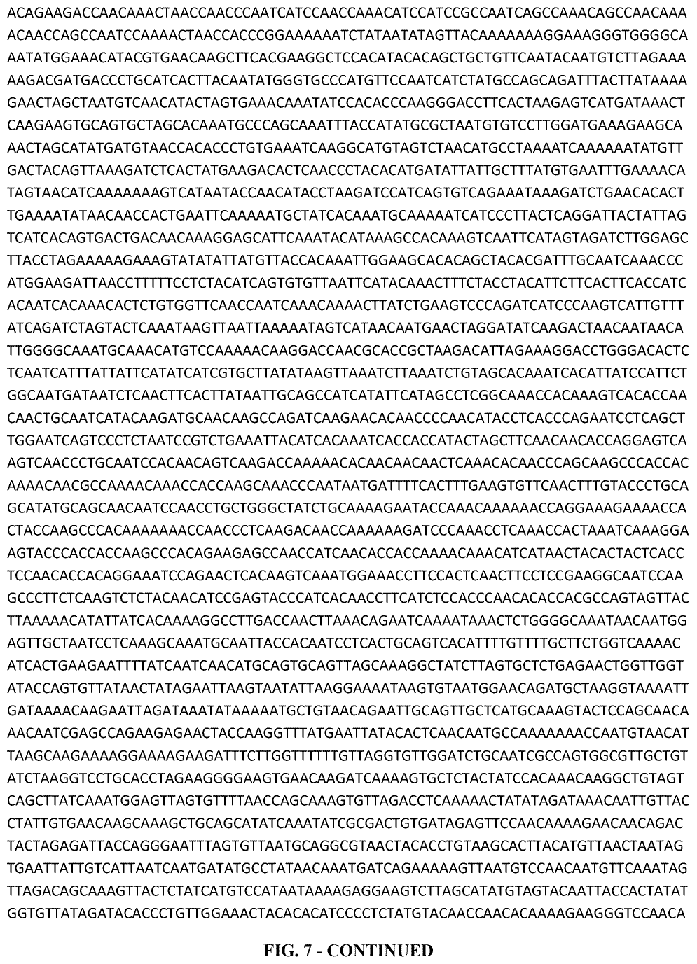

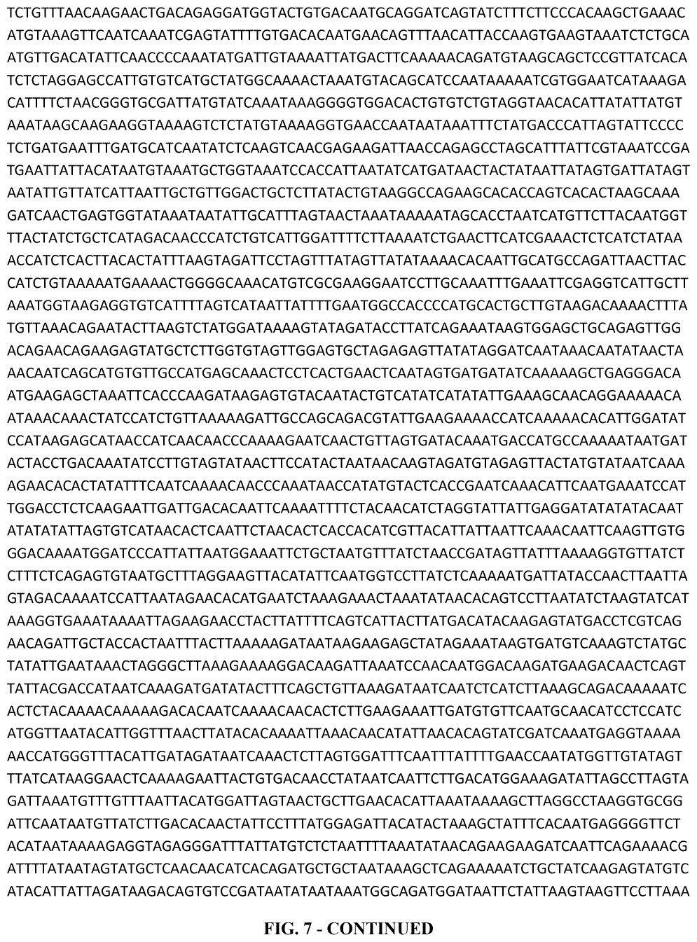

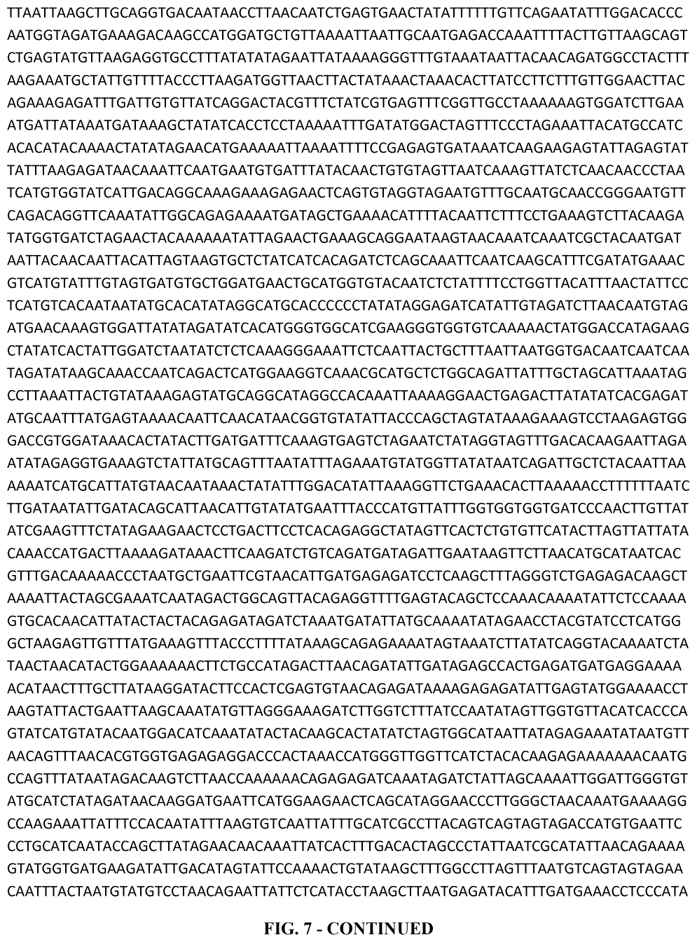

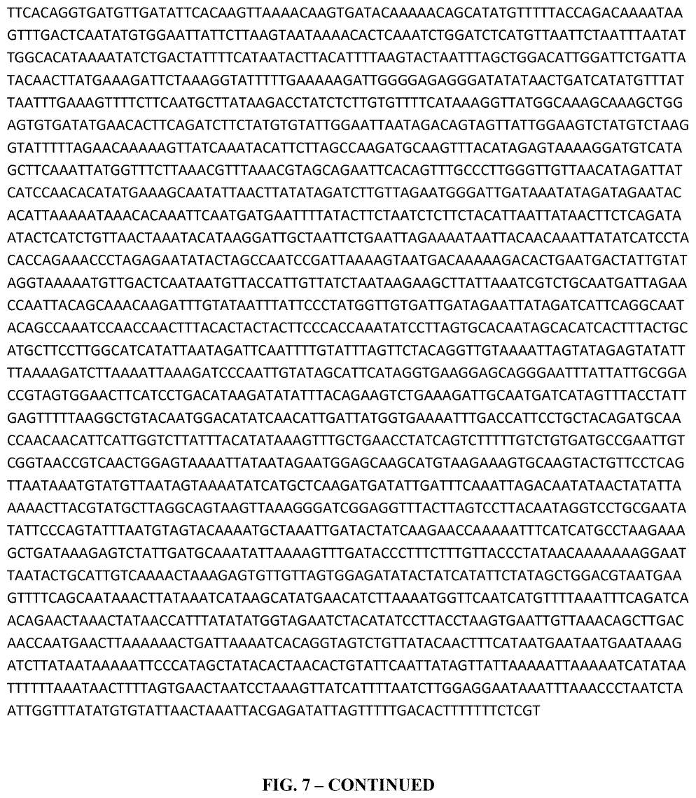

Thus, in the seronegative infants and children, RSV MEDI ΔM2-2 was highly restricted in replication yet more immunogenic than the previous lead live attenuated RSV vaccine candidate rA2 cp248/404/1030ΔSH (nucleotide sequence provided as SEQ ID NO: 2 and shown in FIG. 7), and the RSV MEDI ΔM2-2 vaccine was protective yet primed for substantial anamnestic response to RSV.

Accordingly, in some embodiments, the RSV particle exhibits restricted replication (i.e. low replication as illustrated in Example 2) in the subjects. The term restricted replication refers to a lower degree of replication of the virus particle which leads to an attenuated phenotype of the virus. The restriction of replication may be measured or determined using known methods. For example, in some embodiments, the mean peak group post-vaccination viral infectivity titer in nasal wash specimens (irrespective of day, collected and assayed by the methods of this invention) from an exemplary group of vaccinated children is less than 2.5 log₁₀ PFU/ml of nasal wash, and preferably less than or equal to 1.5-2.0 log₁₀ PFU/ml nasal wash, whereas in comparison vaccination of a comparable group with the test particle rA2 cp248/404/1030ΔSH would yield a mean peak group viral infectivity titer that is approximately equal to or greater than 2.0-3.0 log₁₀ PFU/ml nasal wash. In some embodiments, the frequency or rate of shedding of infectious vaccine virus (i.e., the percentage of the test group from whom infectious vaccine virus is detected) detected by culture following vaccination of an exemplary group of young children, using a dose of approximately 5.0 log₁₀ PFU and following the methods of this invention, is less than approximately 80%, or less than approximately 70%, or about 60%.

In some embodiments, the RSV particle exhibits enhanced immunogenicity in the subjects (as compared, for example, to other known RSV vaccine candidates), in that it exhibits greater vaccine take in the subjects (more subjects show immune response to the vaccine) and/or it elicits higher antibody titers in the subjects. i), The immunogenicity may be measured or determined using known methods. For example, in some embodiments, if an exemplary group of vaccinees is given a dose (such as 5.0 log₁₀ PFU) of the attenuated RSV particle of this invention versus a second, comparable group that received the same dose of a second test attenuated RSV particle with a similar level of attenuation, the mean group titer of serum RSV-neutralizing antibodies, detected by an assay such as the complement-enhanced 60% plaque reduction neutralization assay of this invention, that develops in response to the vaccinations is significantly greater (as determined by standard statistical tests) in the case of the particle of this invention than the test particle. In some embodiments, the immune response (e.g., serum RSV-neutralizing antibody tier) induced by the RSV particle may be higher than that induced by a second test attenuated RSV particle in a comparable test group even if the later particle is less attenuated. In some embodiments, the titer of serum RSV-neutralizing antibodies (sampled and assayed by the methods of this invention) induced by vaccination of an exemplary group of young children with the RSV particle of this invention is not statistically different than that obtained by natural infection (i.e., with a natural, unattenuated RSV) in a comparable group. In some embodiments, the subjects exhibit anamnestic response to a subsequent wild type RSV infection.

That a single dose regimen was sufficient to provide protective effect was a surprising result, as the standard practice in the art has been to administer multiple doses, or at a minimum at least two or three doses, of RSV vaccines in a single vaccination season to provide protective effect. For instance, Karron et al., J. of Infectious Diseases 191:1093-1103 described clinical evaluation of two recombinant RSV candidate viruses rA2cp248/404ΔSH and rA2cp248/404/1030ΔSH in children and infants. Karron et al. concluded that rA2cp248/404ΔSH was not suitable for infants, given its level of replication in RSV-seronegative children. Further, while rA2cp248/404/1030ΔSH exhibited appropriate levels of attenuation, the virus shows limited antibody response in young infants and multiple doses of it, in excess of two, would be required to show any protective effect. Similarly, Wright et al., 2000, J. of Infectious Diseases 182:1331-42 tested the recombinant RSV candidate vaccine cpts-248/404 in a phase 1 trial, and concluded that because the second dose of vaccine did infect two out of three children not infected by the first dose, a schedule of two doses at a minimum of RSV vaccine would be required for immunization. Consistent with this, Malkin et al. 2013, PLOS ONE 8 (10): e77104 and Bernstein et al. 2012, Pediatr. Infect. Dis. J. 31:109-114 evaluated recombinant RSV virus vaccines in healthy, RSV-seronegative children 5 or 6 months to <24 months of age by administration of three doses of the vaccine. See also, Murphy and Collins 2002, J. Clin. Invest. 110:21-27, describing that a desirable immunization sequence employing live-attenuated RSV and PIV vaccines would be administration of RSV and PIV3 vaccines together as a combined vaccine given two or more times; Collins and Murphy 2002, Virology 296:204-211; stating that a live RSV vaccine would be administered two or three times during the first year of life to boost immunity, and Murphy et al. 1994, Virus Research 32:13-36, stating that immunization against RSV will require multiple doses of vaccine to achieve the level of immunity needed to prevent serious disease of the lower respiratory tract.

This expectation of the need for multiple doses is further exemplified by the rotavirus vaccines in current use. Rotavirus resembles RSV in being an RNA virus that infects and causes disease on mucosal surfaces, in this case the enteric tract. RSV and rotavirus are widely considered to share similar challenges in immunizing a mucosal surface in a young child with a live attenuated virus. The RotaTeg rotavirus vaccine in current use is given in three doses at 2, 4, and 6 months of age, and the Rotarix rotavirus vaccine in current use is given in two doses at 2 and 4 months of age. As another aspect, some studies with RSV suggest that the multiple-dose strategy needed to induce adequate protection may face another obstacle. For example, Malkin et al. 2013, PLOS ONE 8 (10): e77104, Karron et al., J. of Infectious Diseases 191:1093-1103, and Wright et al., 2000, J. of Infectious Diseases 182:1331-42 found that, although the seroresponse to the first dose of attenuated RSV was not high, it was highly restrictive to subsequent dose(s) of the attenuated strain (since a vaccine strain is already highly attenuated, even a small amount of immunity can be highly restrictive). As a result of the high level of restriction, the subsequent dose(s) of the live vaccines were poorly immunogenic. Thus, there can be an inherent limitation to the strategy of multiple doses for a live vaccine-namely, that the subsequent doses of a live vaccine may be too restricted to be immunogenic. This exemplified that the ability to induce a substantial immune response with a single dose of a live vaccine is highly advantageous.

Accordingly in one embodiment, the present invention comprises a method of vaccinating against RSV by administration of a single dose in a single vaccination season of the RSV particles described above. Reference herein to a method of vaccinating comprising administration of a single dose means a single dose in a single vaccination season which excludes a second dose in that vaccination season. In some embodiments of the invention, a single dose means a single dose during the lifetime of the subject. The single dose administration is sufficient to provide protective effect in infants and children, and a second dose is neither required nor administered.

The single dose of the composition comprises an “immunogenically effective dose” which is sufficient to induce or enhance the subject's immune response capabilities against RSV. In all subjects, the precise amount of RSV vaccine administered and the timing of administration will be determined by various factors, including the subject's state of health and weight, the mode of administration, the nature of the formulation, etc. Dosages generally range from about 10^{3.0} (3.0 log₁₀) to about 10^{7.0} (7.0 log₁₀) plaque forming units (“PFU”) of virus per subject.

Thus, in some embodiments, the dosage ranges from 10^{3.0} (3.0 log₁₀) to about 10^{7.0} (7.0 log₁₀) PFU of virus per subject. In some embodiments, the dosage ranges from 10^{4.0} (4.0 log₁₀) to about 10^{6.0} (6.0 log₁₀) PFU of virus per subject. In some embodiments, the dosage ranges from 10^{5.0} (5.0 log₁₀) to about 10^{6.0} (6.0 log₁₀) PFU of virus per subject.

In some embodiments, the single dose of the composition comprises about 10^{7.0} PFU of the RSV particles. In some embodiments, the single dose of the composition comprises about 10^{6.0} PFU of the RSV particles. In some embodiments, the single dose of the composition comprises about 10^{5.0} PFU of the RSV particles. In some embodiments, the single dose of the composition comprises about 10^{4.0} PFU of the RSV particles. In some embodiments, the single dose of the composition comprises about 10^{3.0} PFU of the RSV particles.

In some embodiments of the invention, the human subject is an infant or child. In some embodiments, the subject is less than about 36 months of age. In some embodiments, the subject is less than about 30 months of age. In some embodiments, the subject is less than about 24 months of age. In some embodiments, the subject is less than about 18 months of age. In some embodiments, the subject is less than about 12 months of age. In some embodiments, the subject is less than about 9 months of age. In some embodiments, the subject is less than about 8 months of age. In some embodiments, the subject is less than about 7 months of age. In some embodiments, the subject is less than about 6 months of age. In some embodiments, the subject is less than about 5 months of age. In some embodiments, the subject is less than about 4 months of age. In some embodiments, the subject is less than about 3 months of age. In some embodiments, the subject is less than about 2 months of age. In some embodiments, the subject is less than about 1 month of age. In some embodiments, the subject is less than about 30 days of age. In some embodiments, the subject is less than about 20 days of age. In some embodiments, the subject is less than about 15 days of age. In some embodiments, the subject is less than about 10 days of age. In some embodiments, the subject is a new born.

The composition may be administered by any suitable route, including but not limited to, intranasal, intratracheal, subcutaneous, intramuscular, transdermal, topical, oral or the like. In some embodiments, it may be administered intranasally, subcutaneously or, intramuscularly. In some embodiments, it may be administered intranasally. In some embodiments, it may be administered to the upper respiratory tract.

The administration may be performed via any suitable delivery mechanisms. For example, the composition may be delivered via drops, spray, aerosol delivery, powder, injection, topical formulation, oral inoculation, or the like.

In some embodiments, the composition may be administered intranasally via nasal drops, nasal spray (for example, using a device similar to Flumist), aerosol delivery, nasal powder or the like. Examples of possible nasal delivery modalities are provided in Djupesland. 2013, Drug Deliv. And Transl. Res. 3:42-62) and are expressly incorporated herein. In some embodiments, the composition may be administered intranasally via nasal drops. In some embodiments, the composition may be administered intranasally via nasal spray.

In some embodiments, the subject may be administered the composition intranasally in a physiologically acceptable diluent or carrier. This has the advantage of simplicity and safety compared to parenteral immunization with a non-replicating vaccine. It also provides direct stimulation of local respiratory tract immunity, which plays a major role in resistance to RSV. Further, this mode of vaccination effectively bypasses the immunosuppressive effects of RSV-specific maternally-derived serum antibodies, which typically are found in the very young. Also, while the parenteral administration of RSV antigens can sometimes be associated with immunopathologic complications, this has never been observed with a live virus. The nasal drops may be administered into one nostril or both nostrils. Thus, a part of the single dose of the composition may be administered in each nostril. In some embodiments, the single dose is administered in about 0.2 ml to about 1 ml total volume per subject. In some embodiments, the single dose is administered in about 0.5 ml total volume per subject.

In some embodiments, the composition may comprise the RSV particles and a physiologically acceptable carrier and/or adjuvant. Useful carriers are well known in the art, and include buffered solutions at neutral or physiological pH. Examples of suitable carriers include without limitation, buffered saline or Leibowitz L15 medium.

The resulting aqueous solutions may be packaged for use as is, or in frozen form that is thawed prior to use, or lyophilized, the lyophilized preparation being combined with a sterile solution prior to administration, as mentioned above. Alternatively, lyophilized preparations may be delivered to one or both nostrils as a powder (Djupesland. 2013, Drug Deliv. And Transl. Res. 3:42-62). The compositions may contain pharmaceutically acceptable auxiliary substances as required to approximate physiological conditions, which include, but are not limited to, pH adjusting and buffering agents, tonicity adjusting agents, wetting agents and the like, for example, sodium acetate, sodium lactate, sodium chloride, potassium chloride, calcium chloride, sucrose, magnesium sulfate, phosphate buffers, HEPES (4-(2-hydroxyethyl)-1-piperazineethanesulfonic acid) buffer, sorbitan monolaurate, and triethanolamine oleate. Acceptable adjuvants include incomplete Freund's adjuvant, aluminum phosphate, aluminum hydroxide, or alum, which are materials well known in the art. Preferred adjuvants also include Stimulon™ QS-21 (Aquila Biopharmaceuticals, Inc., Worchester, Mass.), MPL™ (3-O-deacylated monophosphoryl lipid A; RIBI ImmunoChem Research, Inc., Hamilton, Mont.), and interleukin-12 (Genetics Institute, Cambridge, Mass.).

Upon immunization with a RSV vaccine composition, the host responds to the vaccine by producing antibodies specific for RSV virus proteins, e.g., F and G glycoproteins. In addition, innate and cell-mediated immune responses are induced, which can provide antiviral effectors as well as regulating the immune response. As a result of the vaccination the host becomes at least partially or completely immune to RSV infection, or resistant to developing moderate or severe RSV disease, particularly of the lower respiratory tract.

In some embodiments, the present invention comprises a composition comprising the RSV particles described above and a pharmaceutically acceptable carrier. In some embodiments, the present invention comprises a composition comprising the RSV particles described above suspended in Leibowitz medium. In further embodiments, the present invention comprises a kit that includes the composition comprising the RSV particles described above and a pharmaceutically acceptable carrier.

To propagate a RSV virus for vaccine use and other purposes, a number of cell lines which allow for RSV growth may be used. RSV grows in a variety of human and animal cells. Preferred cell lines for propagating attenuated RS virus for vaccine use include DBSFRhL-2, MRC-5, and Vero cells. Highest virus yields are usually achieved with epithelial cell lines such as Vero cells. Cells are typically inoculated with virus at a multiplicity of infection ranging from about 0.001 to 1.0, or more, and are cultivated under conditions permissive for replication of the virus, e.g., at about 30-37° C. and for about 3-10 days, or as long as necessary for virus to reach an adequate titer. Temperature-sensitive viruses often are grown using 32° C. as the “permissive temperature.” Virus is removed from cell culture and separated from cellular components, typically by well-known clarification procedures, e.g., centrifugation, and may be further purified as desired using procedures well known to those skilled in the art.

While some embodiments of the present invention have been described, it is apparent that modifications and adaptations of those embodiments will occur to those skilled in the art. It is to be expressly understood, however, that such modifications and adaptations are within the scope of the present invention, as set forth in the following exemplary claims. The examples and figures are provided for the purpose of illustration and are not intended to limit the scope of the present invention. Each publication or other reference disclosed herein is incorporated herein by reference in its entirety, to the extent that there is no inconsistency with the present disclosure.

### EXAMPLES

Example 1. This example describes the generation of the recombinant RSV MEDI ΔM2-2 vaccine.

As previously described, in the recombinant RSV vaccine with an M2-2 deletion, a complementary DNA (cDNA) fragment of 234 nucleotides containing the 78 C-terminal codons of the M2-2 ORF was removed from the antigenomic cDNA encoding the wild-type RSV A2 strain. The sequence encoding the first 12 codons of the M2-2ORF, that is, the region that mostly overlaps the M2-1 ORF, was maintained. The resulting plasmid was used for the recovery of the drug substance at MedImmune. Clinical trial material (CTM) was manufactured in qualified Vero cells at Meridian Life Science Inc. Sequence analysis confirmed that the seed virus and Final Drug Product, RSV Lot no. 002A, were of identical sequence, including the three polymorphisms. The RSV MEDI ΔM2-2 CTM was supplied to the clinical site as a frozen concentrate with a mean infectivity titer of 10^{7.0} PFU/ml. CTM was stored at −70° C. and diluted to dose on-site using Leibovitz L15 medium. L15 medium was also used as the placebo.

Example 2. This example describes the clinical evaluation of the RSV MEDI ΔM2-2.

### Isolation, Quantitation, and Characterization of Virus

NWs were performed using a nasal bulb syringe and 15 to 20 ml of Lactated Ringer's solution. NWs were snap-frozen on site and stored at −80° C. An aliquot of each NW was rapidly thawed and tested for infectivity by plaque assay in HEp-2 cells as previously described. Titers of vaccine virus are expressed as the number of PFU per milliliter of NW fluid. Specimens that were culture-negative were assigned a titer of 0.6 log₁₀ PFU/ml.

Shedding of vaccine virus was also quantified by RT-qPCR amplification of the RSV matrix (M) protein. The following primers and probes were used: forward primer, 5′-gcaaatatggaaacatacgtgaacaa-3′, reverse primer, 5′-GGCACCCATATTGTAAGTGATGCA-3′, and probe, 5′-cttcacgaaggctccacata-3′. The assay was performed using the AgPath-ID One-Step RT-PCR kit and the 7300 Fast real-time PCR system (Applied Biosystems). Copy numbers of the gene of interest were calculated using a plasmid DNA standard curve for the RSV M gene. Analyses were performed using 7500 Fast System SDS software (Applied Biosystems). The limit of detection by RT-qPCR was 1.7 log₁₀ copy numbers/ml; therefore, PCR-negative samples were assigned a titer of 1.7 log₁₀ copy numbers/ml.

To determine whether the vaccine virus was genetically stable, the presence of the M2-2 deletion was verified by sequence analysis of NW isolates obtained from seronegative vaccinees at the time of peak viral shedding. Vaccine virus was isolated by one passage of NW fluid on Vero cells. RT-PCR was performed on total extracted RNA using RSV-specific primers, and consensus sequences of a region corresponding to nucleotides 7997 to 8817 of an RSV A2 reference sequence were generated (Genbank accession no. M74568, spanning the M2-2 deletion). In one case, only a shorter RT-PCR fragment could be obtained for sequence analysis of a region corresponding to nucleotides 8004 to 8695 of the reference sequence.

### Immunologic Assays

Serologic specimens: RSV MEDI ΔM2-2 study. Sera were obtained to measure antibodies to RSV before inoculation, about 1 month after inoculation in adults and seropositive children and about 2 months after inoculation in seronegative children. To measure serum antibody responses during the surveillance period, sera were also obtained from seronegative children between 1 October and 31 October of the calendar year in which the child was enrolled and between 1 April and 30 April of the following calendar year. Thus, adults and seropositive children each had two serum specimens obtained, and seronegative children each had three or four serum specimens obtained, depending on the time of enrollment.

Serologic specimens: Previous study of RSVcp248/404/1030/ΔSH. To compare antibody responses to RSV MEDI ΔM2-2 and the previously evaluated RSVcp248/404/1030/ΔSH in RSV-naïve children, paired pre- and post-vaccination specimens from eight RSV-seronegative children who had received 10^{5.3} PFU of RSVcp248/404/1030/ΔSH in an earlier study (8) were retested for neutralizing antibodies to RSV as described below.

Antibody assays. Sera from all subjects were tested for antibodies to RSV by 60% complement-enhanced plaque reduction neutralization assay and for IgG antibodies to the RSV F glycoprotein by ELISA. The ELISA was performed as previously described, except that the RSV F, provided by Novavax Inc., was a purified baculovirus-expressed protein; 20 ng per well was used in the assay. For the neutralizing antibody assay, the starting dilution was 1:10, and for the ELISA, the starting dilution was 1:200 for adult sera and 1:50 for pediatric sera. The PRNT and RSV F IgG titer are expressed as reciprocal log₂. Antibody responses were defined as ≥4-fold increase in titer in paired specimens.

### Data Analysis

Infection was defined as either the detection of vaccine virus by culture or rRT-qPCR or a ≥4-fold rise in RSV serum neutralizing antibody or in RSV F serum antibody. The mean peak titer of vaccine virus shed (log₁₀ PFU/ml) was calculated for infected vaccinees only. The neutralizing antibody and RSV F IgG reciprocal titers were transformed to log₂ values for calculation of mean log₂ titers, and Student's t test was used to compare mean peak viral titers and antibody titers between groups. Rates of illness among vaccinees and placebo recipients were compared by the two-tailed Fisher's exact test.

### Study Population, Study Design, and Clinical Trial Oversight

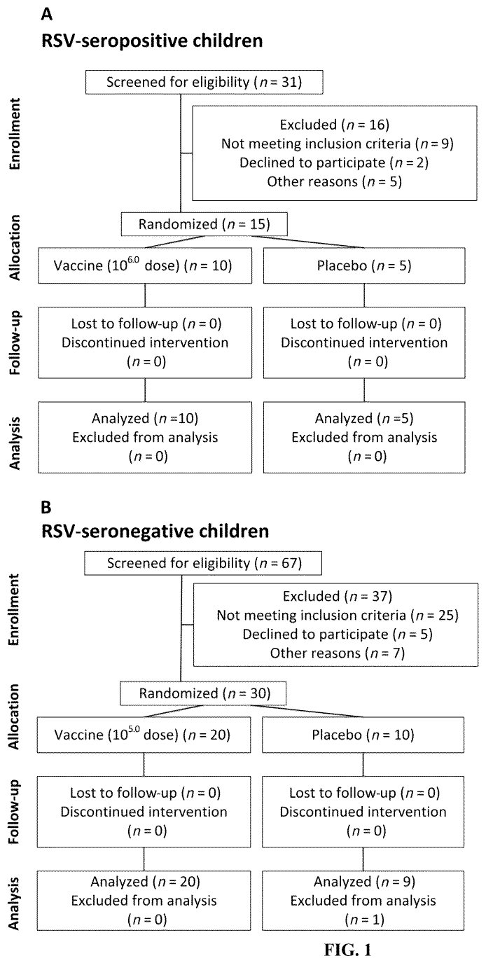

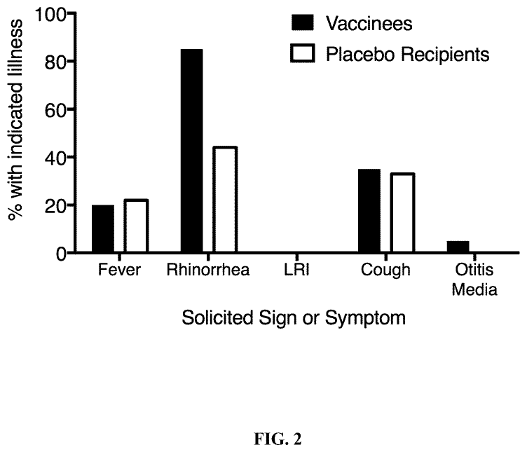

This phase 1 trial was conducted at the Center for Immunization Research (CIR), Johns Hopkins Bloomberg School of Public Health, and Seattle Children's Research Institute between September 2011 and March 2014. The RSV MEDI ΔM2-2 vaccine was evaluated sequentially in (i) adults who were not screened for RSV serostatus (but who all proved to be RSV seropositive), (ii) RSV-seropositive children ages 15 to 59 months, and (iii) RSV seronegative children ages 6 to 24 months. Studies in adults were conducted as open-label trials, with all subjects receiving vaccine. Studies in children were conducted as randomized, double-blind, and placebo controlled trials, with subjects randomized 2:1 to receive vaccine or placebo (FIG. 1). Randomization was performed in blocks of three (two vaccinees and one placebo recipient) to permit unblinding and analysis of adverse events throughout the study, which was done when each group of three subjects completed the initial follow-up period (about 28 days for RSV-seropositive children and about 56 days for RSV-seronegative children). To maintain the study blind, randomization and preparation of the study agent (vaccine or placebo) were performed by study personnel who were not involved in the clinical assessment of study subjects. Vaccine and placebo were each administered in a volume of 0.5 ml as nose drops (about 0.25 ml per nostril). Adults and RSV-seropositive vaccinees received 10^{6.0} PFU of vaccine; RSV-seronegative vaccinees received 10^{5.0} PFU of vaccine. Written informed consent was obtained from study participants (adults) or from the parents or guardians of study participants (children) before enrollment. These studies were conducted in accordance with the principles of the Declaration of Helsinki and the Standards of Good Clinical Practice (as defined by the International Conference on Harmonization). The study was performed under NIAID-held Investigational New Drug applications (BB-IND no. 14763) and was reviewed by the U.S. Food and Drug Administration. The clinical protocol, consent forms, and Investigators' Brochure were developed by CIR and NIAID investigators and were reviewed and approved by the Western Institutional Review Board, and the NIAID Office of Clinical Research Policy and Regulatory Operations (OCRPRO). Clinical data were reviewed by CIR and NIAID investigators and by the Data Safety Monitoring Board of the NIAID Division of Clinical Research.

Clinical assessment: Acute phase. For adults and RSV-seropositive children enrolled in this trial, clinical assessments and NWs were performed on study day 0 (the day of vaccination, with the NW performed before inoculation) and on days 3 to 7 and 10 after inoculation. After day 10, illness data (adverse events and reactogenicity events) were collected through day 28, with additional physical examinations performed and NWs obtained in the event of LRI. All LRIs were defined as SAEs, regardless of severity. For seronegative children, clinical assessments and NWs were performed on study days 0, 3, 5, and 7 and on days 10, 12, 14, 17, 19, 21, and 28±1 day. After the last scheduled NW, illness data were obtained for seronegative children through day 56, with physical examinations performed and additional NWs obtained in the event of LRI. Titers of vaccine virus in NW specimens were determined as described below. Fever, URI (rhinorrhea or pharyngitis), cough, LRI, and OM were defined as previously described (8). When illnesses occurred, NWs were tested for adventitious agents by rRT-PCR (Fast-track Diagnostics).

Clinical assessment: Surveillance. RSV-seronegative children were monitored for symptomatic medically attended RSV-associated illness in this study as previously described (8). In brief, families were contacted weekly between 1 November and 31 March to determine whether MAARIs had occurred, which were defined as fever, URI, LRI, or OM. For each illness episode, a clinical assessment was performed, and a NW obtained for quantitative viral testing by rRT-qPCR and culture as described below.

### Study Participants

RSV MEDI ΔM2-2 was sequentially evaluated in 15 adults, 15 RSV seropositive children, and 30 RSV-seronegative infants and children. This vaccine was evaluated in an open label trial in adults and in randomized, placebo-controlled, double-blind trials in the pediatric populations (FIG. 1). All adults received 10^{6.0} plaque-forming units (PFU) of vaccine; 10 RSV-seropositive children received 10^{6.0} PFU of vaccine and 5 received placebo; and 20 RSV-seronegative vaccinees received 10^{5.0} PFU of vaccine and 10 received placebo. The mean age of adult participants was 36 years (range, 20 to 49 years); of RSV-seropositive participants, 37 months (range, 18 to 59 months); and of RSV-seronegative participants, 13 months (range, 6 to 24 months). Of the 60 participants, 48% were female, 53% white, 33% black, and 14% described as of mixed race.

### Infectivity and Adverse Events

During the 28-day postimmunization reporting period, 3 of 15 vaccinated adults developed respiratory illnesses [hoarseness (1), pharyngitis (1), and rhinorrhea and cough (1)], but none shed vaccine virus or had rises in serum RSV antibody titers. Upper respiratory illness [(URI); rhinorrhea] was observed in 4 of 10 vaccinated RSV-seropositive children during this 28-day period (Table 1); each symptomatic child had rhinovirus or enterovirus detected in nasal wash (NW) at the time of illness. As in the adults, none of the seropositive children shed vaccine virus, which is consistent with attenuation.

URI (rhinorrhea), cough, and febrile illnesses occurred frequently in RSV-seronegative vaccinees and placebo recipients during the 28 days after immunization (Supplemental Table 1 and FIG. 2). The rates of fever (20% versus 22%) and cough (35% versus 33%) were comparable in vaccinees and placebo recipients (Supplemental Table 1 and FIG. 2). URI occurred more often in seronegative vaccinees than in placebo recipients (85% versus 44%), but this difference was not significant and all episodes were of grade 1 (mild) severity (Supplemental Table 1 and FIG. 2). None of the children experienced LRI, and one vaccinee had otitis media (OM). Other viruses were detected frequently in symptomatic children, including rhinovirus, enterovirus, parechovirus, adenovirus, coronavirus, bocavirus, and parainfluenza viruses types 1 and 4.

Kawasaki disease was reported as a serious adverse event (SAE) in a seronegative vaccinee. Vaccine virus was detected in this child's NW by culture on study days 5, 7, and 10 and by real-time reverse transcription quantitative polymerase chain reaction (rRT-qPCR) on study days 3 to 12, and the child had rhinorrhea on study days 8 to 11. The child was otherwise without symptoms until study day 32 when she became febrile. She subsequently developed a rash and oral lesions that were consistent with Kawasaki disease and was hospitalized on study day 35. The child was treated with aspirin and intravenous immuno-globulin (IGIV) and recovered without sequelae. The SAE was judged to be unrelated to the study product on the basis of the interval between infection with vaccine virus and onset of symptoms, as well as the absence of any known association between RSV and Kawasaki disease.

Transmission of vaccine virus occurred between a seronegative vaccine and a placebo recipient who were 13-month-old twin siblings. Twin A (the vaccinee) had vaccine virus detected in NW on study days 5, 7, and 10 and rhinorrhea on study days 8 to 11. Twin B (the placebo recipient) had vaccine virus detected in NW on study days 14 and 17, accompanied by a temperature of 38.1° C. on day 17 (infected placebo recipient, Supplemental Table 1). The peak titer of vaccine virus shed by each child was 2.0 log₁₀ PFU/ml. Sequence analysis revealed that the vaccine virus shed by both children retained the deletion of the M2-2 ORF. Illness, replication, and immunogenicity data from this infected placebo recipient are reported as a separate line item in Tables 1 and 2, and surveillance data from this placebo recipient were excluded from the analysis. These data indicate a major advantage of the MEDI ΔM2-2 vaccine. Specifically, RNA viruses are well known to have a high mutation rate with regard to single nucleotide changes, such that attenuating point mutations are readily subject to de-attenuation. In contrast, gene deletions such as the ΔM2-2 mutation are not known to be subject to de-attenuation. Thus, in this example, the MEDI ΔM2-2 virus was able to transmit to a second individual in a situation where there is very extensive personal contact, such as between young twins, but the virus remained genetically stable and attenuated, and the transmitted virus replicated to only low titer without disease and induced immunity in the twin.

### Replication and Genetic Stability of RSV MEDI ΔM2-2

As noted above, vaccine virus was not detected by viral culture in NW obtained from adults and RSV-seropositive children, consistent with the restriction in replication expected for live attenuated RSV vaccines in RSV-experienced populations.

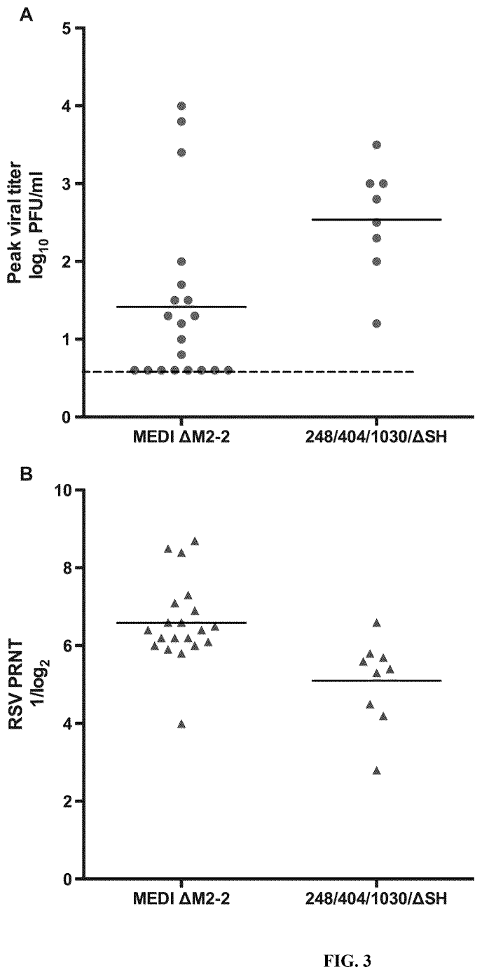

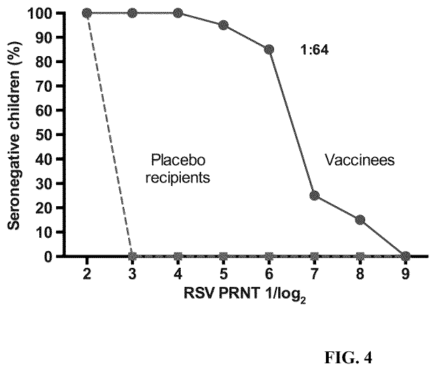

In contrast, vaccine virus was detected in NW by culture in 12 of 20 RSV-seronegative vaccinees and by RTqPCRin 17 of 20 RSV-seronegative vaccinees (Supplemental Table 2 and FIG. 3A; the 3 vaccinees who were negative by RT-qPCR also were negative by culture). The mean peak titer of virus detected by culture in these children was 1.5 log₁₀ PFU/ml. This was significantly lower than that detected in NW from seronegative recipients of rA2 cp248/404/1030/ΔSH (2.5 log₁₀ PFU/ml, P=0.005; FIG. 3A), a previously studied vaccine candidate that was well tolerated and immunogenic in seronegative infants and children (8). Partial sequence analysis of NW isolates obtained at the peak of vaccine shedding from 15 RSV-seronegative vaccinees verified the presence of the M2-2 deletion. PCR products for sequence analysis could not be obtained from four seronegative vaccinees from whom infectious vaccine virus was not recovered and from one vaccine with low peak titer (1.3 log₁₀ PFU/ml).

### Antibody Responses to RSV MEDI ΔM2-2.

A single RSV-seropositive vaccinee had a rise in RSV F serum immunoglobulin G (IgG) titer (Table 2). Thus, none of the adults and one RSV-seropositive vaccinee had evidence of infection with vaccine virus, which is consistent with attenuation. In addition, a seropositive placebo recipient had a rise in both RSV F serum IgG and RSV-neutralizing serum antibody titers [measured as plaque reduction neutralization titer (PRNT)], which likely indicated an infection with a circulating wildtype RSV that was not detected during the post-inoculation follow-up period.

Nineteen of 20 RSV-seronegative children developed neutralizing serum antibody responses, and 18 of 20 developed RSV F serum IgG antibody responses after vaccination (Table 2 and FIG. 4). In seronegative children, the mean post-vaccination PRNT was 6.6 log₂, or 1:97 (Table 2); 85% achieved PRNT ≥6.0 log₂, or 1:64 (FIG. 4). There was no apparent relationship between the magnitude of vaccine virus shedding and the magnitude of the antibody response. Mean PRNTs were significantly higher in seronegative recipients of MEDI ΔM2-2 than in recipients of rA2 cp248/404/1030/ΔSH (6.6 log₂ versus 5.1 log₂, or 1:97 versus 1:34; P=0.002; FIG. 3B). With the exception of the twin to whom vaccine virus was transmitted, none of the RSV-seronegative placebo recipients developed a rise in RSV PRNT.

RSV Surveillance in Seronegative Children with Respect to RSV Related Illness and Antibody Response

Surveillance for medically attended acute respiratory illness (MAARI) was conducted for the seronegative cohort during the RSV season (1 November to 31 March) after vaccine administration, and pre- and post-surveillance sera were collected and tested for PRNT as described above. MAARI was frequent, with 15 episodes occurring in seven vaccinees and 10 episodes occurring in six placebo recipients. However, only three instances of RSV-associated MAARI were detected: RSV subgroup A-associated illness occurred in one placebo recipient (rhinorrhea, cough, and wheezing), and RSV subgroup B-associated illness occurred in one vaccinee (rhinorrhea and cough) and one placebo recipient (rhinorrhea and fever).

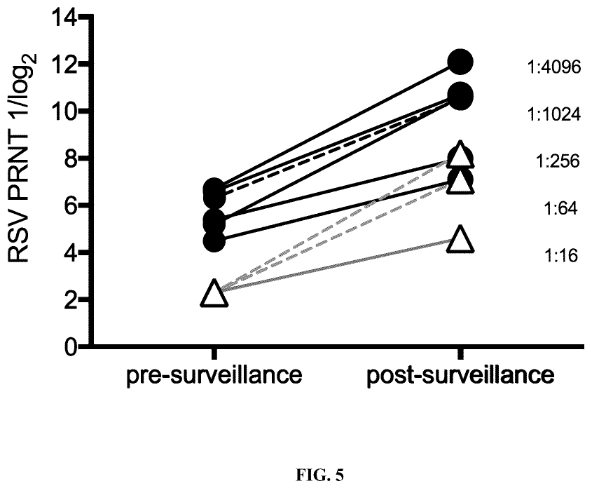

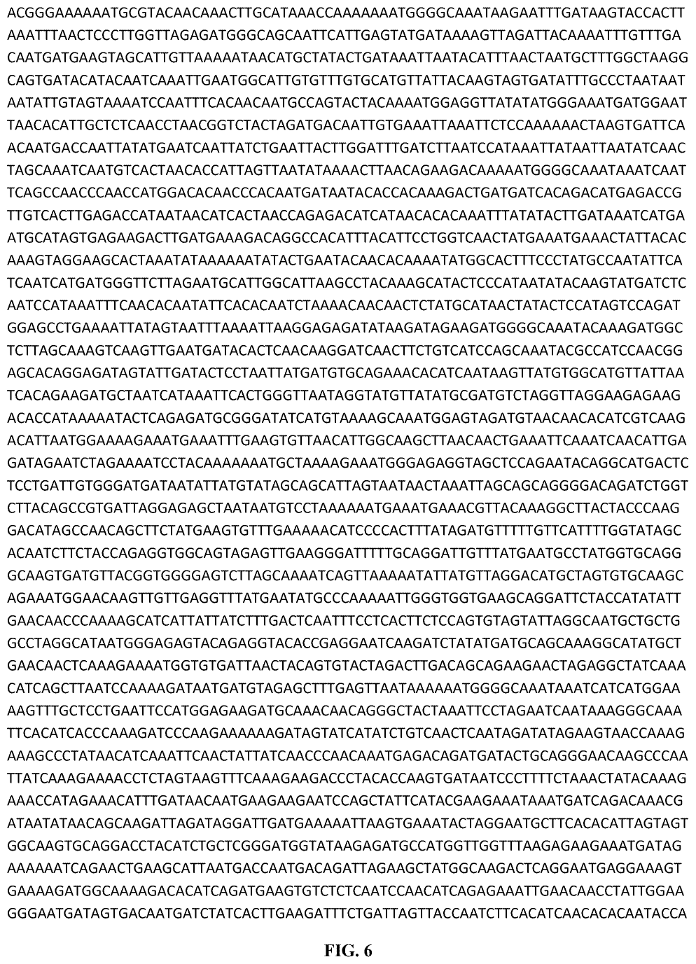

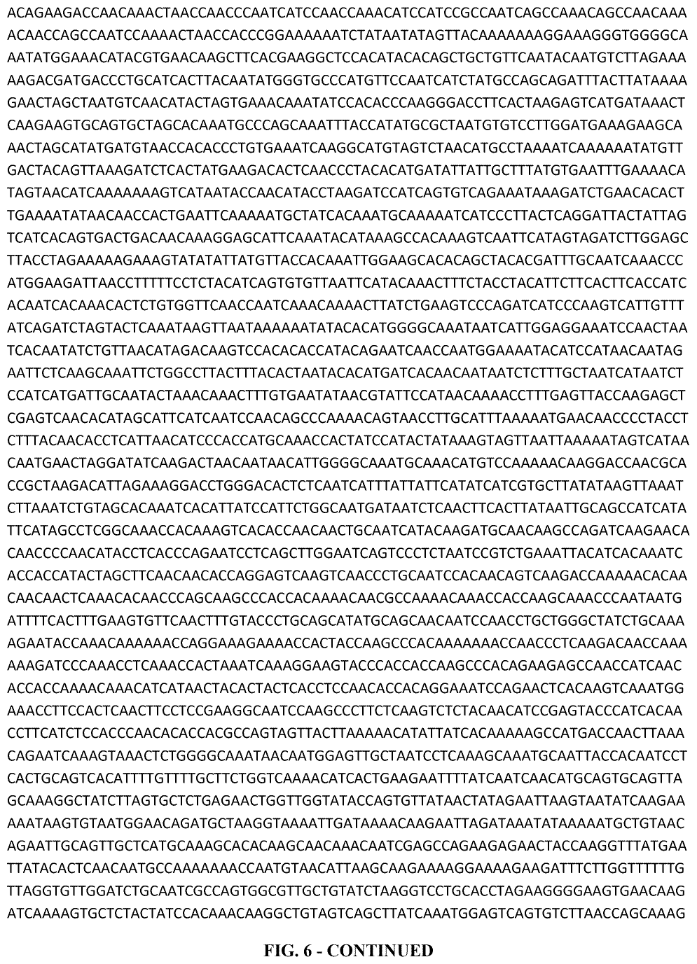

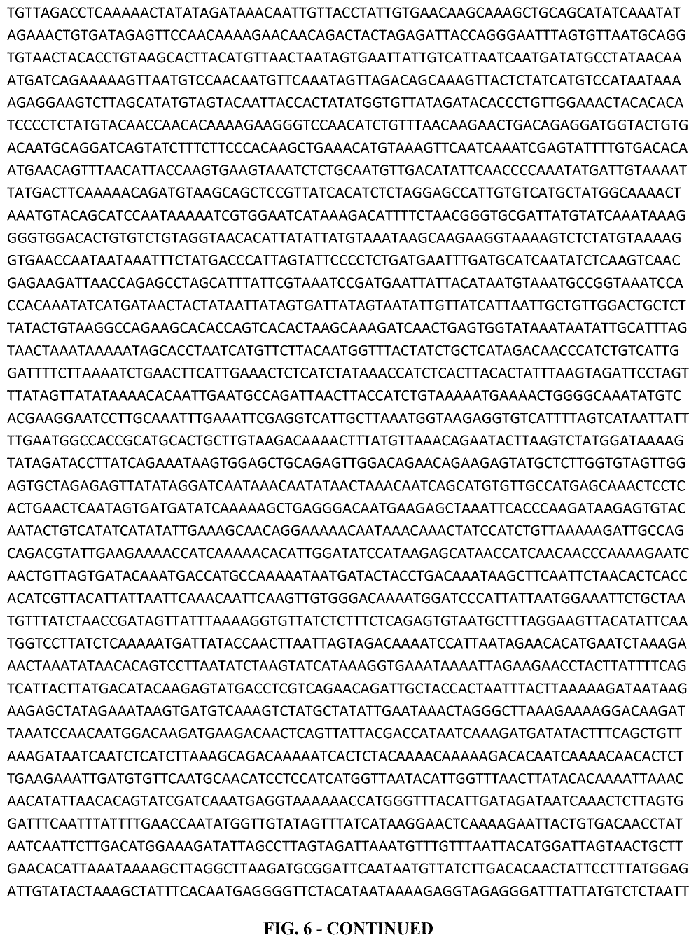

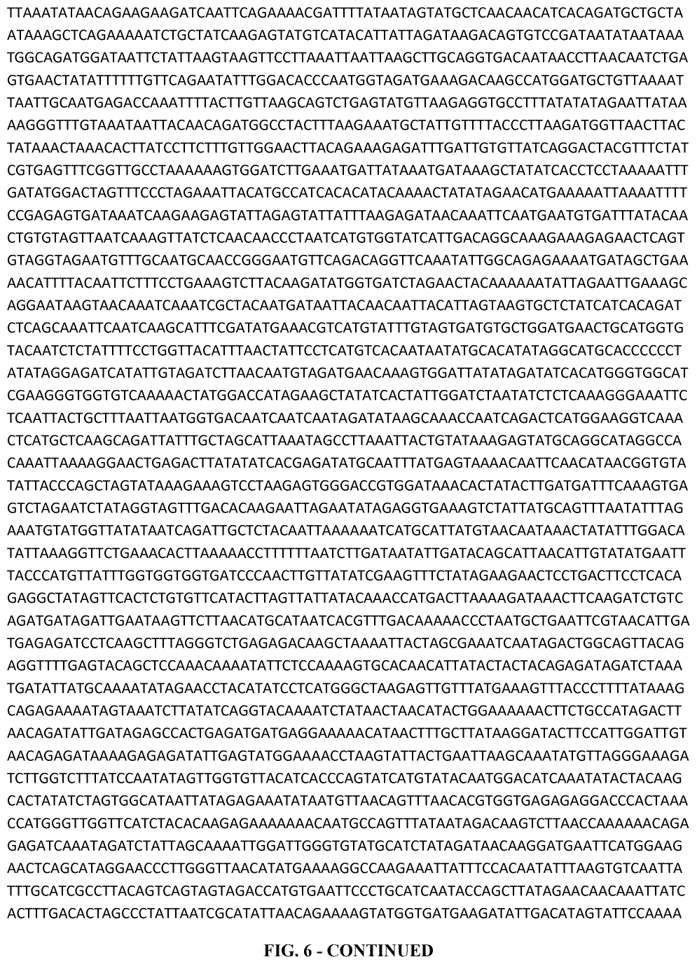

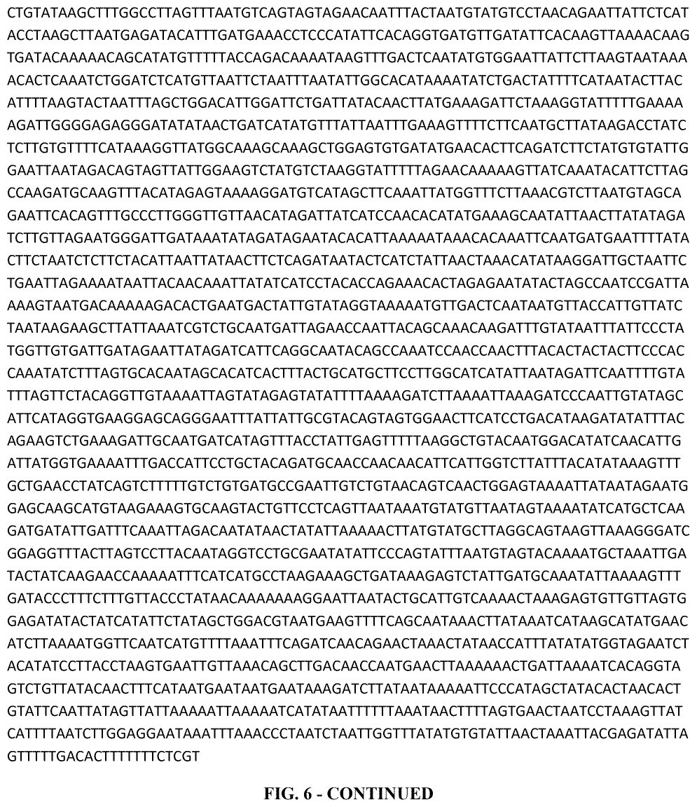

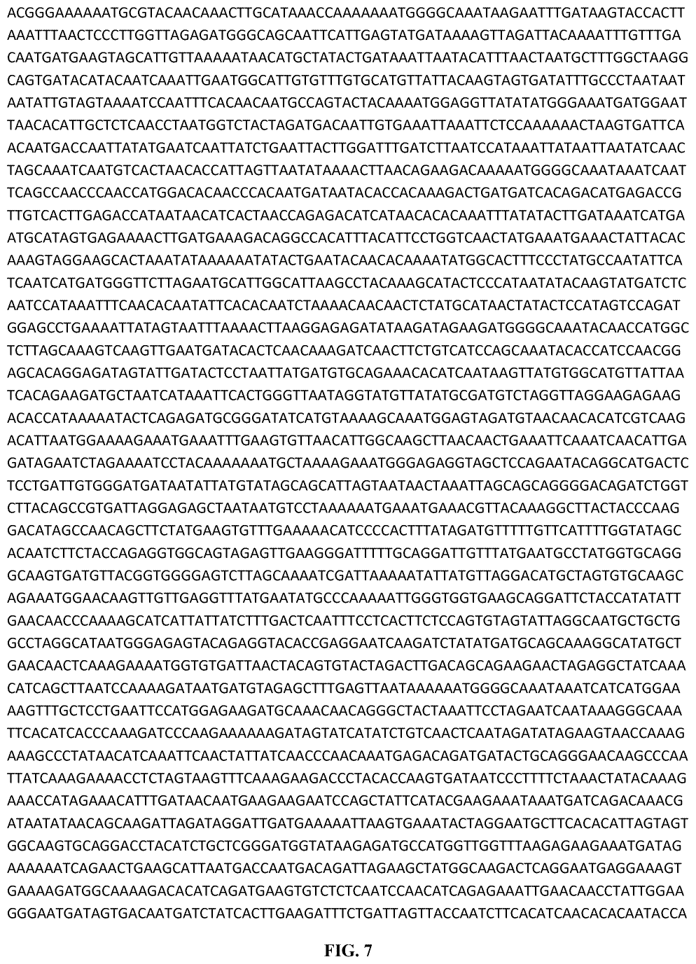

Despite infrequent RSV MAARI, ≥4-fold increases in RSV PRNT were detected in post-surveillance sera from three placebo recipients and six vaccinees, including the three subjects in whom wild-type RSV was detected. The mean post-surveillance log₂ PRNT in the three placebo recipients in whom rises were observed (6.6 log₂) was identical to the mean log₂ PRNT observed in the vaccine recipients after vaccination (Table 2). Thus, the mean RSV PRNT achieved after a single dose of RSV MEDI ΔM2-2 in RSV-seronegative children was the same as that achieved after a primary wild-type RSV infection. Notably, the post-surveillance geometric mean PRNT in the six vaccinated children in whom rises were observed were significantly greater than those achieved in the placebo recipients after natural infection (9.8 log₂ versus 6.6 log₂, or 1:891 versus 1:97, P=0.046; FIG. 5). One of the six vaccinees (subject F5 35, supplementary tables) did not shed vaccine virus and did not have a rise in antibody titer after vaccination, suggesting that he did not have a vaccine “take” and experienced a primary immune response to infection with wild-type RSV. The post-surveillance geometric mean PRNT among the remaining five vaccinees with an initial vaccine take was 10.3 log₂ or 1:1261. Three of these five children achieved post-surveillance serum neutralizing antibody titers of >1:1000, indicating that potent anamnestic responses occurred after wild-type RSV infection in vaccine-primed individuals (FIG. 5).

These data suggest that a number of vaccine recipients had clinically inapparent wild-type RSV infections during the surveillance period that boosted the RSV PRNT greater than nine fold (on average) over the titers achieved after immunization. For vaccine recipients without a significant rise in PRNT, the mean reciprocal log₂ titer was 6.5 (1:91) after immunization and 6.3 (1:79) after the surveillance period, indicating that a neutralizing antibody response was sustained for at least 5 to 12 months after immunization.

TABLE 1
Replication of vaccine virus and clinical assessments in
adults, RSV-seropositive, and RSV-seronegative children.

| | | | | Viral Detection | | | |
|---|---|---|---|---|---|---|---|
| | | | | (culture) | | Viral Detection | |
| | | | | | Peak | (qPCR) | |
| | | | | | titer | Peak | |
| | | | | | mean | copy | |
| | Dose | | | % | (SD) | number | |
| | (log₁₀ | No. of | % | shedding | log₁₀ | log₁₀ | |
| Subjects | PFU/ml) | subjects | infected | virus^{†} | PFU/ml | (SD) | Fever |
| Adults | | | | | | | |
| Vaccinees | 6.0 | 15 | 0 | 0 | 0.6 (0.0) | NT | 0 |
| Seropositive | | | | | | | |
| children | | | | | | | |
| Vaccinees | 6.0 | 10 | 10 | 0 | 0.6 (0.0) | NT | 0 |
| Placebo | Placebo | 20 | 20 | 0 | 0.6 (0.0) | NT | 0 |
| recipients | | | | | | | |
| Seronegative | | | | | | | |
| children | | | | | | | |
| Vaccinees | 5.0 | 20 | 95 | 85 | 1.5 (0.9) | 3.4 (0.9) | 20 |
| Infected | Placebo | 1 | 100 | 100 | 2.0 (0.0) | 3.3 (0.0) | 100 |
| placebo | | | | | | | |
| recipient^{‡} | | | | | | | |
| Placebo | Placebo | 9 | 0 | 0 | 0.6 (0.0) | 1.7 (0.0) | 22 |
| recipients | | | | | | | |

| | % with indicated illness* | | | | | |
|---|---|---|---|---|---|---|
| | | | | | Respiratory | |
| | | | | | or febrile | |
| Subjects | URI | LRI | Cough | OM | illness | Other |
| Adults | | | | | | |
| Vaccinees | 20 | 0 | 7 | 0 | 20 | 27 |
| Seropositive | | | | | | |
| children | | | | | | |
| Vaccinees | 40 | 0 | 0 | 0 | 40 | 20 |
| Placebo | 0 | 0 | 0 | 0 | 0 | 60 |
| recipients | | | | | | |
| Seronegative | | | | | | |
| children | | | | | | |
| Vaccinees | 85 | 0 | 35 | 5 | 85 | 45 |
| Infected | 100 | 0 | 0 | 0 | 100 | 0 |
| placebo | | | | | | |
| recipient^{‡} | | | | | | |
| Placebo | 44 | 0 | 33 | 0 | 67 | 78 |
| recipients | | | | | | |

NT, not tested.
*Illness abbreviations and definitions are as described in the text. URI was defined as rhinorrhea, pharyngitis, or hoarseness, and LRI was defined as wheezing, rhonchi, or rales, or having been diagnosed with pneumonia or laryngotracheobronchitis (croup).
^{†}% Shedding vaccine virus as detected by culture and/or rPT-qPCR. The limit of detection of vaccine virus was 0.6 log₁₀ PFU/ml by culture, and 1.7 log₁₀ copy numbers/ml by rRT-qPCR.
^{‡}Data from the seronegative placebo recipient, as described in the text.

TABLE 2
Replication of vaccine virus and antibody responses in adults, RSV-seropositive children, and RSV-seronegative children.

| | | | | | Serum RSV neutralizing | | | Serum IgG ELISA | | |
|---|---|---|---|---|---|---|---|---|---|---|
| | Dose | | | | antibody* [mean(SD)] | | | RSV F* [mean(SD)] | | |
| | (log₁₀ | No. of | | % Shedding | Pre | Post | ≥4-Fold | Pre | Post | ≥4-Fold |
| Subjects | PFU/ml) | subjects | % infected | virus^{†} | (SD) | (SD) | rise (%) | (SD) | (SD) | rise (%) |
| Adults | | | | | | | | | | |
| Vaccinees | 6.0 | 15 | 0 | 0 | 9.3 (1.1) | 9.6 (0.9) | 0 | 15.1 (1.2) | 15.0 (1.2) | 0 |
| Seropositive | | | | | | | | | | |
| children | | | | | | | | | | |
| Vaccinees | 6.0 | 10 | 10 | 0 | 7.5 (1.9) | 7.7 (1.8) | 0 | 14.2 (2.3) | 14.0 (2.1) | 10 |
| Placebo | Placebo | 5 | 20 | 0 | 8.4 (2.7) | 9.0 (2.9) | 20 | 14.8 (1.1) | 15.6 (1.4) | 20 |
| recipients | | | | | | | | | | |
| Seronegative | | | | | | | | | | |
| children | | | | | | | | | | |
| Vaccinees | 5.0 | 20 | 95 | 85 | 2.7 (0.9) | 6.6 (1.1) | 95 | 7.1 (2.7) | 13.6 (1.6) | 90 |
| Infected | Placebo | 1 | 100 | 100 | 2.3 (0.0) | 7.6 (0.0) | 100 | 5.6 (0.0) | 13.6 (0.0) | 100 |
| placebo | | | | | | | | | | |
| recipient^{‡} | | | | | | | | | | |
| Placebo | Placebo | 9 | 0 | 0 | 2.3 (0.0) | 2.3 (0.0) | 0 | 5.4 (1.5) | 5.1 (1.0) | 0 |
| recipients | | | | | | | | | | |

ELISA, enzyme-linked immunosorbent assay.
*Antibody data are expressed as reciprocal mean log₂ titers.
^{†}% Shedding vaccine virus as detected by culture and/or rRT-qPCR. The limit of detection of vaccine virus was 0.6 log₁₀ PFU/ml.
^{‡}Data from the seronegative placebo recipient, as described in the text.

SUPPLEMENTAL TABLE 1
Clinical responses and viral shedding in seropositive children 12 months to 59 months of age
to 10^{6.0} PFU of live recombinant respiratory syncytial virus vaccine RSV MEDI ΔM2-2 or placebo

| | | | | | | |
|---|---|---|---|---|---|---|
| | | | | Virus shedding | | |
| | | | | (culture) | Adventitious | |
| Date of | Subject | Age/ | | peak log₁₀ | agents | Illness (days) |
| inoculation | No. | months | Inf | titer/mL | (days) | Fever |

Vaccines

| | | | | | | |
|---|---|---|---|---|---|---|
| Nov. 11, 2011 | 17 | 39 | 0 | 0.6 | neg (0) | 0 |
| Dec. 2, 2011 | 18 | 41 | 0 | 0.6 | neg(0)Rhino(10) | 0 |
| Dec. 2, 2011 | 19 | 37 | 0 | 0.6 | neg(0, 10) | 0 |
| Feb. 23, 2012 | 21 | 44 | 0 | 0.6 | neg(0)Adeno(8)Rhino(8, 11 | 0 |
| Mar. 9, 2012 | 22 | 39 | 0 | 0.6 | Rhino(0)neg(5)Entero(6-7) | 0 |
| Mar. 9, 2012 | 23 | 18 | 0 | 0.6 | neg(0, 4, 7)Rhino(3) | 0 |
| Mar. 30, 2012 | 26 | 31 | 0 | 0.6 | 0 | 0 |
| Mar. 30, 2012 | 27 | 44 | 0 | 0.6 | 0 | 0 |
| Apr. 5, 2012 | 28 | 31 | 0 | 0.6 | neg(0)Rhino(11) | 0 |
| Apr. 13, 2012 | 29 | 57 | 0 | 0.6 | 0 | 0 |
| | Mean | 38.1 | 0/10 | 0.6 | | 0/10 |
| | SD % | 10.2 | 0 | 0.0 | | 0 |

Placebo recipients

| | | | | | | |
|---|---|---|---|---|---|---|
| Nov. 11, 2011 | 16 | 21 | 0 | 0.6 | Rhino(0) | 0 |
| Dec. 16, 2011 | 20 | 59 | 0 | 0.6 | 0 | 0 |
| Mar. 23, 2012 | 24 | 27 | 1 | 0.6 | 0 | 0 |
| Mar. 29, 2012 | 25 | 39 | 0 | 0.6 | 0 | 0 |
| Apr. 20, 2012 | 30 | 27 | 0 | 0.6 | 0 | 0 |
| | Mean | 34.6 | 1/5 | 0.6 | | 0/5 |
| | SD % | 15.1 | 20 | 0.0 | | 0 |

| | Illness (days) | | | | | |
|---|---|---|---|---|---|---|
| | | | | | Respiratory | |
| Date of | | | | | or febrile | |
| inoculation | URI | LRI | Cough | OM | illness | Other |
| Vaccines | | | | | | |
| Nov. 11, 2011 | 0 | 0 | 0 | 0 | 0 | 0 |
| Dec. 2, 2011 | 0 | 0 | 0 | 0 | 0 | NC(10-13)¹ |
| Dec. 2, 2011 | 0 | 0 | 0 | 0 | 0 | 0 |
| Feb. 23, 2012 | R(8-12)¹ | 0 | 0 | 0 | 1 | 0 |
| Mar. 9, 2012 | R(1-6)¹ | 0 | 0 | 0 | 1 | 0 |
| Mar. 9, 2012 | R(3-7)¹ | 0 | 0 | 0 | 1 | Epistaxis(5)¹ |
| Mar. 30, 2012 | 0 | 0 | 0 | 0 | 0 | 0 |
| Mar. 30, 2012 | 0 | 0 | 0 | 0 | 0 | 0 |
| Apr. 5, 2012 | R(9-16)¹ | 0 | 0 | 0 | 1 | 0 |
| Apr. 13, 2012 | 0 | 0 | 0 | 0 | 0 | 0 |
| | 4/10 | 0/10 | 0/10 | 0/10 | 4/10 | 2/10 |
| | 40 | 0 | 0 | 0 | 40 | 20 |
| Placebo recipients | | | | | | |
| Nov. 11, 2011 | 0 | 0 | 0 | 0 | 0 | Skin Excoriation (3-10)¹ |
| Dec. 16, 2011 | 0 | 0 | 0 | 0 | 0 | Epistaxis(4)¹ |
| Mar. 23, 2012 | 0 | 0 | 0 | 0 | 0 | 0 |
| Mar. 29, 2012 | 0 | 0 | 0 | 0 | 0 | 0 |
| Apr. 20, 2012 | 0 | 0 | 0 | 0 | 0 | Abd Discomfort(3)¹ |
| | 0/5 | 0/5 | 0/5 | 0/5 | 0/5 | 3/5 |
| | 0 | 0 | 0 | 0 | 0 | 60 |

URI = upper respiratory illness, LRI = lower respiratory illness, OM = otitis media, R = rhinorrhea, NC = nasal congestion, Adeno = adenovirus, Rhino = rhinovirus, Entero = enterovirus, Inf = infected with vaccine virus as determined by shedding of vaccine virus and/or ≥4-fold increase in antibody titers. Parentheses indicate study days on which the event occurred. Superscripted numbers next to the sign or symptom indicate severity grade as described in the text and figure legends. Shedding of vaccine virus was detected by culture (limit of detection = 0.7 log10/mL; culture negative expressed as 0.6 log10/mL). Adventitious agents were detected by multiplex RT-PCR.

SUPPLEMENTAL TABLE 2
Antibody responses of seropositive children 12 to 59
months of age to 106.0 PFU of live recombinant respiratory
syncytial virus vaccine RSV MEDI ΔM2-2 or placebo

| | | | Serum Antibody Responses | | | | | |
|---|---|---|---|---|---|---|---|---|
| | | | RSV Neutralizing | | | IgG ELISA | | |
| | | | Antibody (1/log₂) | | | RSV F (1/log₂) | | |
| | | | | | ≥4-Fold | | | ≥4-Fold |
| Date of | Subject | Age/ | | | rise | | | rise |
| inoculation | No. | months | Pre | Post | (%) | Pre | Post | (%) |

Vaccines

| | | | | | | | | |
|---|---|---|---|---|---|---|---|---|
| Nov. 11, 2011 | 17 | 39 | 7.8 | 7.4 | 0 | 13.6 | 13.6 | 0 |
| Dec. 2, 2011 | 18 | 41 | 7.5 | 8.4 | 0 | 15.6 | 15.6 | 0 |
| Dec. 2, 2011 | 19 | 37 | 5.6 | 5.9 | 0 | 11.6 | 13.6 | 1 |
| Feb. 23, 2012 | 21 | 44 | 8.2 | 7.5 | 0 | 15.6 | 15.6 | 0 |
| Mar. 9, 2012 | 22 | 39 | 5.7 | 5.9 | 0 | 13.6 | 11.6 | 0 |
| Mar. 9, 2012 | 23 | 18 | 5.2 | 5.0 | 0 | 9.6 | 9.6 | 0 |
| Mar. 30, 2012 | 26 | 31 | 9.9 | 9.1 | 0 | 15.6 | 15.6 | 0 |
| Mar. 30, 2012 | 27 | 44 | 7.2 | 8.1 | 0 | 15.6 | 15.6 | 0 |
| Apr. 5, 2012 | 28 | 31 | 11.3 | 11.2 | 0 | 17.6 | 15.6 | 0 |
| Apr. 13, 2012 | 29 | 57 | 7.0 | 8.2 | 0 | 13.6 | 13.6 | 0 |
| | Mean | 38.1 | 7.5 | 7.7 | 0/10 | 14.2 | 14.0 | 1/10 |
| | SD % | 10.2 | 1.9 | 1.8 | 0 | 2.3 | 2.1 | 10 |

Placebo recipients

| | | | | | | | | |
|---|---|---|---|---|---|---|---|---|
| Nov. 11, 2011 | 16 | 21 | 7.8 | 6.9 | 0 | 15.6 | 15.6 | 0 |
| Dec. 16, 2011 | 20 | 59 | 6.3 | 6.2 | 0 | 13.6 | 13.6 | 0 |
| Mar. 23, 2012 | 24 | 27 | 7.2 | 12.8 | 1 | 13.6 | 17.6 | 1 |
| Mar. 29, 2012 | 25 | 39 | 7.5 | 7.8 | 0 | 15.6 | 15.6 | 0 |
| Apr. 20, 2012 | 30 | 27 | 13.0 | 11.3 | 0 | 15.6 | 15.6 | 0 |
| | Mean | 34.6 | 8.4 | 9.0 | 1.5 | 14.8 | 15.6 | 1/5 |
| | SD % | 15.1 | 2.7 | 2.9 | 20 | 1.1 | 1.4 | 20 |

SUPPLEMENTAL TABLE 3
Clinical responses of seronegative children 6 months to 24 months of age to 10^{5.0} PFU
of live recombinant respiratory syncytial virus vaccine RSV MEDI ΔM2-2 or placebo

| Date of | | | | Illness (days) | | |
|---|---|---|---|---|---|---|
| inoculation | Subject No. | Age/Months | Inf | Fever | URI | LRI |

Vaccines

| | | | | | | |
|---|---|---|---|---|---|---|
| May 11, 2012 | 32 | 12 | 1 | 100.1 (9)^{0a} | R(3-15)¹ | 0 |
| May 18, 2012 | 33 | 7 | 1 | 102.5 (24-25)³ | R(7-10)¹ | 0 |
| May 18, 2012 | 34 | 13 | 1 | 104.6 (23-26)³ | R(7-12)¹ | 0 |
| May 25, 2012 | 35 | 13 | 0 | 0 | 0 | 0 |
| Jul. 6, 2012 | 37 | 13 | 1 | 0 | R(24-36)¹ | 0 |
| Jul. 13, 2012 | 38 | 22 | 1 | 0 | R(4-15)¹ | 0 |
| Aug. 10, 2012 | 41 | 14 | 1 | 0 | R(4-13)¹ | 0 |
| Aug. 31, 2012 | 42 | 11 | 1 | 0 | R(7-13)¹ | 0 |
| Sep. 14, 2012 | 44 | 13 | 1 | 0 | R(4-10)¹ | 0 |
| Jun. 28, 2013 | 45 | 15 | 1 | 0 | 0 | 0 |
| May 30, 2013 | 46 | 18 | 1 | 0 | R(5-11)¹ | 0 |
| Aug. 2, 2013 | 48 | 13 | 1 | 0 | R(8-11)¹ | 0 |
| Aug. 23, 2013 | 50 | 12 | 1 | 0 | 0 | 0 |
| Sep. 13, 2013 | 51 | 8 | 1 | 100.8(10)¹ | R(8-11)¹ | 0 |
| Sep. 20, 2013 | 52 | 10 | 1 | 0 | R(6-10)¹ | 0 |
| Sep. 20, 2013 | 54 | 13 | 1 | 0 | R(8-12)¹ | 0 |
| Oct. 24, 2013 | 55 | 20 | 1 | 0 | R(6-7, 9-21)¹ | 0 |
| Oct. 25, 2013 | 56 | 24 | 1 | 0 | R(4-11, 20-28)¹ | 0 |
| Oct. 12, 2012 | 59 | 14 | 1 | 0 | R(6-10)¹ | 0 |
| Aug. 31, 2012 | 60 | 9 | 1 | 0 | R(4-14, 22-29)¹ | 0 |
| | Mean | 13.7 | 19/20 | 4/20 | 17/20 | 0/20 |
| | SD % | 4.4 | 95 | 20 | 85 | 0 |

Placebo recipients

| | | | | | | |
|---|---|---|---|---|---|---|
| May 11, 2012 | 31 | 6 | 0 | 0 | R(1-17)¹ | 0 |
| Jun. 1, 2012 | 36 | 14 | 0 | 102.2 (27-28)₂ | 0 | 0 |
| Jul. 25, 2012 | 39 | 12 | 0 | 101.7(7-8)₂ | R(6-12)¹ | 0 |
| Aug. 3, 2012 | 40 | 9 | 0 | 0 | 0 | 0 |
| Sep. 7, 2012 | 43 | 14 | 0 | 0 | R(4-13)¹ | 0 |
| Aug. 16, 2013 | 49 | 7 | 0 | 0 | 0 | 0 |
| Sep. 20, 2013 | 53 | 14 | 0 | 0 | 0 | 0 |
| Oct. 31, 2013 | 57 | 19 | 0 | 0 | R(1-2, 4-6)¹ | 0 |
| Apr. 5, 2013 | 58 | 12 | 0 | 0 | 0 | 0 |
| | Mean | 11.9 | 1/10 | 2/9 | 4/9 | 0/9 |
| | SD % | 4.0 | 10 | 22 | 44 | 0 |

Placebo recipient infected with vaccine virus

| | | | | | |
|---|---|---|---|---|---|
| 275.47 | 13 | 1 | 100.6(17)¹ | R(8-9)¹ | 0 |

| | Illness (days) | | | |
|---|---|---|---|---|
| Date of | | | Respiratory or | |
| inoculation | Cough | OM | febrile illness | Order |

Vaccines

| | | | | |
|---|---|---|---|---|
| May 11, 2012 | 0 | 0 | 1 | 0 |
| May 18, 2012 | 0 | 0 | 1 | 0 |
| May 18, 2012 | 0 | 0 | 1 | NC(7-10)1, diaper rash(21-24)2 |
| May 25, 2012 | 0 | 0 | 0 | 0 |
| Jul. 6, 2012 | 0 | 0 | 1 | 0 |
| Jul. 13, 2012 | (3-10)¹ | (4-15)² | 1 | NC(1-2)1, ear bruise(22-26)1 |
| Aug. 10, 2012 | 0 | 0 | 1 | emesis(5)1, NC(8-14)1 |
| Aug. 31, 2012 | (7-9)¹ | 0 | 1 | diarrhea(0-1)1, diaper rash(2)2 |
| Sep. 14, 2012 | 0 | 0 | 1 | NC(11-12)1, emesis(20)1, |
| | | | | diarrhea(22-23)1 |
| Jun. 28, 2013 | 0 | 0 | 0 | 0 |
| May 30, 2013 | (9-10)¹ | 0 | 1 | 0 |
| Aug. 2, 2013 | 0 | 0 | 1 | Kawasaki Syndrome (32-47)3; |
| | | | | hospitalization (35-38) |
| Aug. 23, 2013 | 0 | 0 | 0 | 0 |
| Sep. 13, 2013 | (26-32)¹ | 0 | 1 | insomnia(15)1 |
| Sep. 20, 2013 | 0 | 0 | 1 | 0 |
| Sep. 20, 2013 | 0 | 0 | 1 | 0 |
| Oct. 24, 2013 | (9-10, 16-17)¹ | 0 | 1 | 0 |
| Oct. 25, 2013 | 0 | 0 | 1 | malaise(22)1 |
| Oct. 12, 2012 | 0 | 0 | 1 | 0 |
| Aug. 31, 2012 | (4-5)¹ | 0 | 1 | NC(6-14)1 |
| | 7/20 | 1/20 | 17/20 | 9/20 |
| | 35 | 5 | 85 | 45 |

Placebo recipients

| | | | | |
|---|---|---|---|---|
| May 11, 2012 | 0 | 0 | 1 | 0 |
| Jun. 1, 2012 | 0 | 0 | 1 | 0 |
| Jul. 25, 2012 | (5-12, 18-21)¹ | 0 | 1 | swollen eyelid(12)1 |
| Aug. 3, 2012 | (8-9, 13-14)¹ | 0 | 1 | NC(14)1 |
| Sep. 7, 2012 | 0 | 0 | 1 | finger burn(7)1, NC(14-19)1 |
| Aug. 16, 2013 | 0 | 0 | 0 | malaise(11)1 |
| Sep. 20, 2013 | 0 | 0 | 0 | head bump(4)1 |
| Oct. 31, 2013 | (1-2, 22-26)¹ | 0 | 1 | chest congestion(21-25)1, |
| | | | | malaise(0, 2, 7, 24-25)1 |
| Apr. 5, 2013 | 0 | 0 | 0 | insomnia(10)1 |
| | 3/9 | 0/9 | 6/9 | 7/9 |
| | 33 | 0 | 67 | 70 |

Placebo recipient infected with vaccine virus

| | | | |
|---|---|---|---|
| 0 | 0 | 1 | 0 |

URI = upper respiratory illness, LRI = lower respiratory illness, OM = otitis media, R = rhinorrhea, NC = nasal congestion, Inf = infected with vaccine virus as determined by shedding of vaccine virus and/or ≥4-fold increase in antibody titers. Parentheses indicate study days on which the event occured. Superscripted numbers next to the sign or symptom indicate severity grade as described in the text and figure legends.

respiratory syncytial virus vaccine RSV MEDI ΔM2-2 or placebo

| | | | Viral Detection (culture) | | | Viral Detection (RT-qPCT) | | |
|---|---|---|---|---|---|---|---|---|
| Dates of | Subject | Age/ | | Virus Detected | Peak Titer | Virus detected | Peak Copy | |
| Inoculation | No. | Months | Inf | By Culture | Log₁₀ | By PCR | log₁₀ | Adventitious agents (days) |

Vaccinees

| | | | | | | | | |
|---|---|---|---|---|---|---|---|---|
| May 5 11, 2012 | 32 | 12 | 1 | 1 | 3.8 | 1 | 4.5 | neg(0), RSV(3, 5, 6, 10), |
| | | | | | | | | parecho(5), Cor43(6, 10, 11) |
| May 18, 2012 | 33 | 7 | 1 | 1 | 1 | 1 | 1.8 | neg(0, 24, 25, 27), RSV(11), rhino(6) |
| May 18, 2012 | 34 | 13 | 1 | 1 | 1.3 | 1 | 3 | neg(0, 11, 24, 35, 27) |
| May 25, 2012 | 35 | 13 | 0 | 0 | 0.6 | 0 | 1.7 | 0 |
| Jul. 6, 2012 | 37 | 13 | 1 | 0 | 0.6 | 0 | 1.7 | (neg0, 26), rhino(28) |
| Jul. 13, 2012 | 38 | 22 | 1 | 1 | 0.8 | 1 | 3.2 | neg(0), RSV(3, 5, 7, 10), boca(5) |
| Aug. 10, 2012 | 41 | 14 | 1 | 0 | 0.6 | 1 | 3.3 | neg(0), RSV(5, 7, 10, 14) |
| Aug. 31, 2012 | 42 | 11 | 1 | 0 | 0.6 | 1 | 3.1 | neg(0), RSV(10, 12) |
| Sep. 14, 2012 | 44 | 13 | 1 | 1 | 1.5 | 1 | 2.8 | neg(0), RSV(5, 7, 10), rhino(10) |
| Jun. 28, 2013 | 45 | 15 | 1 | 1 | 1.2 | 1 | 4.1 | 0 |
| May 30, 2013 | 46 | 18 | 1 | 1 | 1.5 | 1 | 3.9 | adeno(0, 11), RSV(6, 8) |
| Aug. 2, 2013 | 48 | 13 | 1 | 1 | 2 | 1 | 4.5 | neg(0), RSV(7, 10) |
| Aug. 23, 2013 | 50 | 12 | 1 | 0 | 0.6 | 0 | 1.7 | 0 |
| Sep. 13, 2013 | 51 | 8 | 1 | 1 | 1.3 | 1 | 3.2 | neg(0), RSV(10), rhino(10, 12, |
| | | | | | | | | 14, 19, 27), parecho(19, 27) |
| Sep. 20, 2013 | 52 | 10 | 1 | 0 | 0.6 | 1 | 3.1 | Cor43, parecho(0), RSV(7, 10) |
| Sep. 20, 2013 | 54 | 13 | 1 | 1 | 1.7 | 1 | 3.9 | neg(0), RSV(10, 12) |
| Oct. 24, 2013 | 55 | 20 | 1 | 0 | 0.6 | 1 | 4 | neg(0, 11, 12, 14, 20, 21), |
| | | | | | | | | RSV(7), boca(18), adeno(18) |
| Oct. 25, 2013 | 56 | 24 | 1 | 0 | 0.6 | 1 | 2.9 | neg(0, 12, 28), RSV(5, 7, 10), rhino(21) |
| Oct. 12, 2012 | 59 | 14 | 1 | 1 | 3.4 | 1 | 4 | 0 |
| Aug. 31, 2012 | 60 | 9 | 1 | 1 | 4 | 1 | 5.3 | parecho (11, 12), rhino (25, 28) |
| | Mean | 13.7 | 19/20 | 12/20 | 1.5 | 17/20 | 3.4 | |
| | SD | 4.4 | | | 0.9 | | 0.9 | |
| | % | | 95 | 60 | | 85 | | |

Placebo recipients

| | | | | | | | |
|---|---|---|---|---|---|---|---|
| May 11, 2012 | 31 | 6 | 0 | 0.6 | 0 | 1.7 | rhino(0, 3, 18), neg(10) |
| Jun. 1, 2012 | 36 | 14 | 0 | 0.6 | 0 | 1.7 | neg(0, 27-28) |
| Jul. 25, 2012 | 39 | 12 | 0 | 0.6 | 0 | 1.7 | neg (0, 7, 8, 21), rhino (9, 12) |
| Aug. 3, 2012 | 40 | 9 | 0 | 0.6 | 0 | 1.7 | boca(0), neg(10, 12, 14, 17), rhino(27, 28) |
| Sep. 7, 2012 | 43 | 14 | 0 | 0.6 | 0 | 1.7 | neg(0, 7), entero(5, 10, 14) |
| Aug. 16, 2013 | 49 | 7 | 0 | 0.6 | 0 | 1.7 | 0 |
| Sep. 20, 2013 | 53 | 14 | 0 | 0.6 | 0 | 1.7 | 0 |
| Oct. 31, 2013 | 57 | 19 | 0 | 0.6 | 0 | 1.7 | Para-1(0, 3), neg(5, 7, 27), Para-4(21) |
| Apr. 5, 2013 | 58 | 12 | 0 | 0.6 | 0 | 1.7 | 0 |
| | Mean | 11.9 | 0/9 | 0.6 | 0/9 | 1.7 | |
| | SD | 4.0 | | 0.0 | | 0.0 | |
| | % | | 0 | | 0 | | |

Placebo recipient infected with vaccine virus

| | | | | | | | |
|---|---|---|---|---|---|---|---|
| Aug. 2, 2013 | 47 | 13 | 1 | 2 | 1 | 3.3 | neg(0, 7, 10), RSV(17) |

Adeno = adenovirus, Rhino = rhinovirus, Entero = enterovirus, Inf = infected with vaccine virus as determined by shedding of vaccine virus and/or ≥4-fold increase in antibody titers. Parentheses indicate study days on which the event occurred. Shedding of vaccine virus was detected by culture (limit of detection = 0.7 log10/mL; culture negative expressed as 0.6 log10/mL) and by RT- qPCR (limit of detection = 1.7 log10 copies/mL; PCR negative expressed as 1.7 log10 copies/mL). Adventitious agents were detected by multiplex RT-PCR.

SUPPLEMENTAL TABLE 5
Antibody responses of seronegative children 6 to 24 months of age of 10^{5.0} PFU of
live recombinant respiratory syncytial virus vaccine RSV MEDI ΔM2-2 or placebo

| | | | Viral | Serum Antibody Responses RSV | | | | | |
|---|---|---|---|---|---|---|---|---|---|
| | | | Shedding | Neutralizing Antibody (1/log2) | | | IgG ELISA RSV F (1/log2) | | |
| Date of | Subject | Age/ | Culture+ | | | ≥4 Fold | | | ≥4 Fold |
| inoculation | No. | months | Or PCR+ | Pre | Post | Rise | Pre | Post | Rise |

Vaccines

| | | | | | | | | | |
|---|---|---|---|---|---|---|---|---|---|
| May 11, 2012 | 32 | 12 | 1 | 2.3 | 7.3 | 1 | 4.6 | 13.6 | 1 |
| May 18, 2012 | 33 | 7 | 1 | 3.5 | 6.5 | 1 | 7.6 | 13.6 | 1 |
| May 18, 2012 | 34 | 13 | 1 | 2.3 | 6.2 | 1 | 5.6 | 15.6 | 1 |
| May 25, 2012 | 35 | 13 | 0 | 4.5 | 4.0 | 0 | 11.6 | 11.6 | 0 |
| Jul. 6, 2012 | 37 | 13 | 0 | 2.3 | 6.1 | 1 | 9.6 | 11.6 | 1 |
| Jul. 13, 2012 | 38 | 22 | 1 | 2.3 | 6.4 | 1 | 4.6 | 13.6 | 1 |
| Aug. 10, 2012 | 41 | 14 | 1 | 2.3 | 7.1 | 1 | 4.6 | 15.6 | 1 |
| Aug. 31, 2012 | 42 | 11 | 1 | 2.3 | 6.0 | 1 | 9.6 | 13.6 | 1 |
| Sep. 14, 2012 | 44 | 13 | 1 | 2.3 | 5.9 | 1 | 4.6 | 11.6 | 1 |
| Jun. 28, 2013 | 45 | 15 | 1 | 2.3 | 6.6 | 1 | 4.6 | 13.6 | 1 |
| May 30, 2013 | 46 | 18 | 1 | 2.3 | 6.4 | 1 | 5.6 | 13.6 | 1 |
| Aug. 2, 2013 | 48 | 13 | 0 | 2.3 | 8.5 | 1 | 4.6 | 15.6 | 1 |
| Aug. 23, 2013 | 50 | 12 | 1 | 2.3 | 6.9 | 1 | 9.6 | 13.6 | 1 |
| Sep. 13, 2013 | 51 | 8 | 1 | 2.3 | 5.8 | 1 | 7.6 | 13.6 | 1 |
| Sep. 20, 2013 | 52 | 10 | 1 | 2.3 | 6.6 | 1 | 5.6 | 13.6 | 1 |
| Sep. 20, 2013 | 54 | 13 | 1 | 2.3 | 6.0 | 1 | 4.6 | 13.6 | 1 |
| Oct. 24, 2013 | 55 | 20 | 1 | 5.0 | 8.7 | 1 | 11.6 | 15.6 | 1 |
| Oct. 25, 2013 | 56 | 24 | 1 | 2.3 | 6.2 | 1 | 4.6 | 15.6 | 1 |
| Oct. 12, 2012 | 59 | 14 | 1 | 4.2 | 8.4 | 1 | 11.6 | 13.6 | 1 |
| Aug. 3, 2012 | 60 | 9 | 1 | 2.3 | 6.2 | 1 | 9.6 | 9.6 | 0 |
| | Mean | 13.7 | 17/20 | 2.7 | 6.6 | 19/20 | 7.1 | 13.6 | 18/20 |
| | SD | 4.4 | | 0.9 | 1.1 | | 2.7 | 1.6 | |
| | % | | 85 | | | 95 | | | 90 |

Placebo Recipients

| | | | | | | | | | |
|---|---|---|---|---|---|---|---|---|---|
| May 11, 2012 | 31 | 6 | 0 | 2.3 | 2.3 | 0 | 9.6 | 7.6 | 0 |
| Jun. 1, 2012 | 36 | 14 | 0 | 2.3 | 2.3 | 0 | 4.6 | 4.6 | 0 |
| Jul. 25, 2012 | 39 | 12 | 0 | 2.3 | 2.3 | 0 | 4.6 | 4.6 | 0 |
| Aug. 3, 2012 | 40 | 9 | 0 | 2.3 | 2.3 | 0 | 4.6 | 4.6 | 0 |
| Sep. 7, 2012 | 43 | 14 | 0 | 2.3 | 2.3 | 0 | 4.6 | 4.6 | 0 |
| Aug. 16, 2013 | 49 | 7 | 0 | 2.3 | 2.3 | 0 | 5.6 | 5.6 | 0 |
| Sep. 20, 2013 | 53 | 14 | 0 | 2.3 | 2.3 | 0 | 4.6 | 4.6 | 0 |
| Oct. 31, 2013 | 57 | 19 | 0 | 2.3 | 2.3 | 0 | 4.6 | 4.6 | 0 |
| Apr. 5, 2013 | 58 | 12 | 0 | 2.3 | 2.3 | 0 | 5.6 | 4.6 | 0 |
| | Mean | 11.9 | 0/9 | 2.3 | 2.3 | 0/9 | 5.4 | 5.1 | 0/9 |
| | SD | 4.0 | | 0.0 | 0.0 | | 1.6 | 1.0 | |
| | % | | 0 | | | 0 | | | 0 |

Placebo Recipient infected with vaccine virus

| | | | | | | | | | |
|---|---|---|---|---|---|---|---|---|---|
| Aug. 2, 2013 | 47 | 13 | 1 | 2.3 | 7.6 | 1 | 5.6 | 13.6 | 1 |

SUPPLEMENTAL TABLE 6
RSV neutralizing antibody titers in sera obtained before and after the RSV surveillance period in
RSV seronegative children 6 to 24 months of age who had received RSV MEDI ΔM2-2 vaccine or placebo

| | PRE-VAC | POST-VAC | PRE-SURV | POST-SURV | PRE-VAC | POST-VAC | PRE-SURV | POST-SURV |
|---|---|---|---|---|---|---|---|---|
| | RSV Neut Ab Reciprocal Titer | | | RSV Neut Ab Titer expressed as 1/log₂ | | | | |
| Vaccinee | | | | | | | | |
| Subject# | | | | | | | | |
| 32 | 5 | 109 | 59 | 158 | 2.3 | 6.8 | 5.9 | 7.3 |
| 33 | 5 | 47 | 36 | 1550 | 2.3 | 5.6 | 5.2 | 10.6 |
| 34 | 11 | 59 | 24 | 29 | 3.5 | 5.9 | 4.6 | 4.9 |
| 35 | 27 | 24 | 22 | 135 | 4.8 | 4.6 | 4.5 | 7.1 |
| 37 | 5 | 65 | 45 | 17 | 2.3 | 6.0 | 5.5 | 4.1 |
| 38 | 5 | 77 | 79 | 1443 | 2.3 | 6.3 | 6.3 | 10.5 |
| 41 | 5 | 109 | 114 | 200 | 2.3 | 6.8 | 6.8 | 7.6 |
| 42 | 5 | 82 | 71 | 70 | 2.3 | 6.4 | 6.1 | 6.1 |
| 44 | 5 | 41 | 41 | 250 | 2.3 | 5.4 | 5.4 | 8.0 |
| 45 | 5 | 177 | 123 | 110 | 2.3 | 7.5 | 6.9 | 6.8 |
| 46 | 5 | 120 | 95 | 1702 | 2.3 | 6.9 | 6.6 | 10.7 |
| 48 | 5 | 350 | 350 | 889 | 2.3 | 8.5 | 8.5 | 9.8 |
| 50 | 5 | 141 | 141 | 35 | 2.3 | 7.1 | 7.1 | 5.1 |
| 51 | 5 | 42 | 42 | 25 | 2.3 | 5.4 | 5.4 | 4.6 |
| 52 | 5 | 78 | 78 | 93 | 2.3 | 6.3 | 6.3 | 6.5 |
| 54 | 5 | 40 | 40 | 28 | 2.3 | 5.3 | 5.3 | 4.8 |
| 55 | 29 | 418 | 418 | 373 | 4.9 | 8.7 | 8.7 | 8.5 |
| 56 | 5 | 106 | 106 | 4485 | 2.3 | 6.7 | 6.7 | 12.1 |
| 59 | 21 | 224 | 224 | 100 | 4.4 | 7.8 | 7.8 | 6.6 |
| 60 | 5 | 46 | 46 | 27 | 2.3 | 5.5 | 5.5 | 4.8 |
| Placebo | | | | | | | | |
| Subject# | | | | | | | | |
| 31 | 10 | 5 | 5 | 5 | 3.3 | 2.3 | 2.3 | 2.3 |
| 36 | 5 | 5 | 5 | 24 | 2.3 | 2.3 | 2.3 | 4.6 |
| 39 | 5 | 5 | 5 | 5 | 2.3 | 2.3 | 2.3 | 2.3 |
| 40 | 5 | 5 | 5 | 5 | 2.3 | 2.3 | 2.3 | 2.3 |
| 43 | 5 | 5 | 5 | 289 | 2.3 | 2.3 | 2.3 | 8.2 |
| 49 | 5 | 5 | 5 | 5 | 2.3 | 2.3 | 2.3 | 2.3 |
| 53 | 5 | 5 | 5 | 5 | 2.3 | 2.3 | 2.3 | 2.3 |
| 57 | 5 | 5 | 5 | 135 | 2.3 | 2.3 | 2.3 | 7.1 |
| 58 | 5 | 5 | 5 | 5 | 2.3 | 2.3 | 2.3 | 2.3 |

Placebo recipient infected with vaccine virus

| | | | | | | | | |
|---|---|---|---|---|---|---|---|---|
| 275.47 | 5 | 291 | N/A | 294 | 2.3 | 8.2 | N/A | 8.2 |

RSV neutralizing antibody titers were measured in serum specimens obtained prevaccination (PRE-VAC), at day 56 following vaccination (POST-VAC), within 1 month prior to the beginning of the surveillance period (PRE-SURV), and within one month following the end of the surveillance period (POST-SURV). In instances where the day 56 POST-VAC specimen was obtained within 1 month prior to the beginning of the surveillance period, an additional PRE-SURV specimen was not obtained and results from the single specimen were entered as both POST-VAC and PRE-SURV (indicated in blue).

What is claimed is:

1. A method of vaccinating a human subject against respiratory syncytial virus (RSV), comprising administering to the subject a composition comprising an RSV particle that comprises an RSV genome or antigenome,
wherein the subject is less than about 24 months of age,
wherein the composition is administered in a single dose, and
wherein the RSV genome or antigenome comprises a functional deletion in the M2-2 ORF corresponding to a deletion of nucleotides 8201-8434 in Genbank Accession No. M74568; and optionally comprises a characteristic selected from the group consisting of
i) presence of one or more of, corresponding to the nucleotides in Genbank Accession No. M74568, C4488 in the SH gene, C4491 in the SH gene, A4494 in the SH gene, A4496 in the SH gene, G4497 in the SH gene, and a nucleotide sequence corresponding to nucleotides 4498-4609 in the non-translated region of the SH gene;
ii) presence of G at a nucleotide position corresponding to position 1209 in Genbank Accession No. M74568 encoding an Alanine at position 24 in the N protein;
iii) presence of G at a nucleotide position corresponding to position 779 in Genbank Accession No. M74568 encoding an Arginine at position 51 in the NS2 protein;
iv) presence of one or more nucleotides corresponding to the nucleotides in Genbank Accession No. M74568 selected from the group consisting of: A1 138, GI 139, A561 1, A5615, G5639, T7481 and A7559;
v) presence of one or more nucleotides corresponding to the nucleotides in Genbank Accession No. M74568 selected from the group consisting of: C404 in the NSI gene, G1181 in the N gene, A1937 in the N gene, C62 15 in the F gene, C622 1 in the F gene, T63 86 in the F gene, C7214 in the F gene, G7701 in the M2 gene; T8160 in the M2-2 gene, T8 1 66 in the M2-2 gene, T8 1 78 in the M2-2 gene, A8 1 97 in the M2-2 gene, G8198 in the M2-2 gene, T 10514 in the L gene, A 13633 in the L gene, and T 13 900 in the L gene;
vi) presence of T at a nucleotide position corresponding to position 1099 in Genbank Accession No. M74568; and
vii) a combination of the above.

2. The method of claim 1, wherein the single dose of the composition comprises about 10^{3.0} to about 10^{7.0} plaque-forming units (PFU) of RSV particles.

3. The method of claim 1, wherein the single dose of the composition comprises about 10^{4.0} plaque-forming units (PFU) of RSV particles.

4. The method of claim 1, wherein the single dose is administered in about 0.2 ml to about 1 ml total volume per subject.

5. The method of claim 1, wherein the single dose is administered in about 0.5 ml total volume per subject.

6. The method of claim 1, wherein the RSV genome or antigenome comprises at least one characteristic listed in (i)-(vi).

7. The method of claim 1, wherein the RSV genome or antigenome comprises all characteristics listed in (i)-(vi).

8. The method of claim 1, wherein the RSV genome or antigenome comprises a nucleotide sequence that is at least 90% identical to SEQ ID NO: 1.

9. The method of claim 1, wherein the RSV genome or antigenome comprises the nucleotide sequence of SEQ ID NO: 1.

10. The method of claim 1, wherein the RSV particle is live attenuated.

11. The method of claim 1, wherein the RSV genome or antigenome comprises one or more dimorphisms selected from the group consisting of: (i) A/G dimorphism at nucleotide position 285 in the NS 1 gene resulting in a mixture of amino acid assignments S/G at amino acid position 63 (ii) C/T dimorphism at nucleotide position 900 in the NS2 gene; and (iii) T/G dimorphism at nucleotide position 4311 in the SH gene resulting in a mixture of amino acid assignments NIK at amino acid position 3.

12. The method of claim 1, wherein the subject is less than about 18 months of age.

13. The method of claim 1, wherein the route of administration is intranasal, intramuscular or subcutaneous.

14. The method of claim 1, wherein the composition is administered via nasal drops.

15. The method of claim 1, wherein the composition is administered via nasal spray.

16. The method of claim 1, wherein the composition is administered via nasal powder.

17. The method of claim 1, wherein the RSV particle exhibits restricted replication in the subject.

18. The method of claim 1, wherein the RSV particle exhibits enhanced immunogenicity in the subject.

19. The method of claim 1, wherein the subject exhibits anamnestic response to wild type RSV infection.

20. A method for vaccinating a human subject against respiratory syncytial virus (RSV) infection, comprising administering to the subject a composition comprising an RSV particle that comprises an RSV genome or antigenome,
wherein the subject less than about 24 months of age,
wherein the composition is administered intranasally in a single dose, and
wherein the RSV genome or antigenome comprises:
i) functional deletion in the M2-2 ORF corresponding to a deletion of nucleotides 8201-8434 in Genbank Accession No. M74568;
ii) presence of, corresponding to the nucleotides in Genbank Accession No. M74568, C4488 in the SH gene, C4491 in the SH gene, A4494 in the SH gene, A4496 in the SH gene, G4497 in the SH gene, and a nucleotide sequence corresponding to nucleotides 4498-4609 in the non-translated region of the SH gene;
iii) presence of G at a nucleotide position corresponding to position 1209 in Genbank Accession No. M74568 encoding an Alanine at position 24 in the N protein;
iv) presence of G at a nucleotide position corresponding to position 779 in Genbank Accession No. M74568 encoding an Arginine at position 51 in the NS2 protein;
v) presence of nucleotides corresponding to the nucleotides in Genbank Accession No. M74568 selected from the group consisting of: A1 138, GI 139, A561 1, A5615, G5639, T7481 and A7559;
vi) presence of nucleotides corresponding to the nucleotides in Genbank Accession No. M74568 selected from the group consisting of: C404 in the NSI gene, GI 181 in the N gene, A1939 A1937 in the N gene, C62 15 in the F gene, C622 1 in the F gene, T63 86 in the F gene, C72 1 4 in the F gene, G7701 in the M2 gene; T8160 in the M2-2 gene, T8166 in the M2-2 gene, T8 1 78 in the M2-2 gene, A8 1 97 in the M2-2 gene, G8 1 98 in the M2-2 gene, T 10514 in the L gene, A 13633 in the L gene, and T 13 900 in the L gene and
vii) presence of T at a nucleotide position corresponding to position 1099 in Genbank Accession No. M74568.
